(19) United States
(12) Patent Application Publication
(10) Pub. No.: US 2023/0372273 A1
(43) Pub. Date: Nov. 23, 2023

(54) IONIC LIQUID COMPOSITIONS FOR SKIN DISEASES AND CONDITIONS

(71) Applicant: CAGE BIO, INC., San Carlos, CA (US)

(72) Inventors: Nitin JOSHI, Redwood City, CA (US); Marina SHEVACHMAN, Los Gatos, CA (US); Kevin W. GELSTON, San Francisco, CA (US); Abhirup MANDAL, Forth Worth, TX (US)

(21) Appl. No.: 18/030,177

(22) PCT Filed: Oct. 4, 2021
(86) PCT No.: PCT/US2021/053429

§ 371 Date: Apr. 4, 2023

Related U.S. Application Data
(60) Provisional application No. 63/087,769, filed on Oct. 5, 2020.

(51) Int. Cl. A61K 31/201; A61K 9/06; A61K 31/14; A61K 47/38; A61K 47/10; A61K 31/355; A61P 17/00

(52) U.S. Cl. CPC A61K 31/201; A61K 9/06; A61K 31/14; A61K 47/38; A61K 47/10; A61K 31/355; A61P 17/00

(57) ABSTRACT

Disclosed herein, in certain embodiments, are gel formulations and compositions comprising anionic liquid comprising a choline cation and fatty acid anion. In some embodiments, the ionic liquid is further formulated for topical administration. In some embodiments, the gel formulations and compositions provided herein are useful for treating skin conditions and disorders such as rosacea.

### BACKGROUND OF THE DISCLOSURE

Rosacea is a common inflammatory skin disorder affecting over 15 million people worldwide. The primary symptoms of rosacea are erythema (abnormal redness of the skin), telangiectasia (visible red lines due to abnormal dilation of capillary vessels), pimple-like eruptions (papules) and pustules. Currently available topical treatments have limited effectiveness and cannot treat all symptoms, particularly erythema. Surgery, such as the laser elimination of blood vessels, is typically a last resort, but may be prescribed if other treatments are ineffective. Thus, there remains a need for topical formulations for treatment of rosacea and its symptoms.

### SUMMARY OF THE DISCLOSURE

In an aspect, there is provided a pharmaceutical gel composition, comprising:
(i) an ionic liquid having a cationic component and an anionic component; and
(ii) at least one pharmaceutically carrier,
wherein the composition is non-irritating to skin, and wherein the composition comprises about 25% to about 45% by weight of the ionic liquid.

In an aspect, there is provided a pharmaceutical gel composition, comprising:
(i) an ionic liquid having a cationic component and an anionic component; and
(ii) at least one pharmaceutically carrier,
wherein the composition is non-irritating to skin, and wherein the composition comprises about 35% to about 45% by weight of the ionic liquid.

In another aspect, there is provided a pharmaceutical gel composition, comprising:
(i) a deep eutectic solvent having a cationic component and an anionic component; and
(ii) at least one pharmaceutically carrier,
wherein the composition is non-irritating to skin, and wherein the deep eutectic solvent has a melting point lower than the melting points of the cationic component and anionic component individually.

In some embodiments, at least one of the anionic component and cationic component is irritating to the skin when applied in the absence of the other component.

In some embodiments, the anionic component is bistriflimide, a geranate, an oleate, a hexanoate, dodecyldimethyl ammonia propane sulfonate, N-lauryl sarcosinate, or a geraniolate.

In some embodiments, the cationic component is benzyl pyridinium, benzyl dimethyl dodecyl ammonium, a choline cation, phosphonium, benzethonium, or a phosphonium.

In some embodiments, the phosphonium is a tetraalkyl phosphonium of structural Formula (I): PR₄, wherein R is a substituted or unsubstituted alkyl group.

In some embodiments, the cationic component is a choline cation.

In some embodiments, the anionic component is a geranate anion.

In some embodiments, the cationic component and the anionic component are in a molar ratio ranging from about 1:1 to about 1:2 (cationic component to anionic component).

In some embodiments, the cationic component and the anionic component are in a molar ratio of about 1:2 (cationic component to anionic component).

In some embodiments, the composition comprises from about 1.0% to about 90% of choline geranate by weight.

In some embodiments, the composition comprises from about 20% to about 60% of choline geranate by weight.

In some embodiments, the composition comprises from about 35% to about 45% of choline geranate by weight.

In some embodiments, the composition comprises about 40% of choline geranate by weight.

In some embodiments, the composition further comprises a pH adjuster, skin conditioner, dye, fragrance, gelling agent, humectant, emollient, antioxidant, preservative, solvent, co-solvent, or combinations thereof.

In some embodiments, the fragrance is D-limonene.

In some embodiments, the fragrance is present in the composition an amount of from about 0.05% to about 5% by weight.

In some embodiments, the fragrance is present in the composition an amount of from about 0.2% to about 1% by weight.

In some embodiments, the gelling agent is hydroxyethyl cellulose.

In some embodiments, the gelling agent is present in the composition an amount of from about 0.05% to about 5% by weight.

In some embodiments, the gelling agent is present in the composition an amount of from about 0.3% to about 1.5% by weight.

In some embodiments, the humectant is glycerin.

In some embodiments, the humectant is present in the composition an amount of from about 0.5% to about 5% by weight.

In some embodiments, the humectant is present in the composition an amount of from about 1% to about 2% by weight.

In some embodiments, the composition further comprises emollients.

In some embodiments, the composition further comprises propylene glycol.

The propylene glycol is present in the composition an amount of from about 5% to about 20% by weight.

In some embodiments, the composition further comprises water.

In some embodiments, the composition has a viscosity of from about 5,000 cP to about 25,000 cP.

In some embodiments, the composition has a pH of from about 6.0 to about 8.0.

In some embodiments, the composition is stable for up to at least about 6 months at a temperature of about 40° C.

In some embodiments, the composition is stable for up at least about 5 days at 80° C.

In some embodiments, the composition is stable to UV-A light for at least about 3 days.

In some embodiments, the composition is placed on or in an applicator or dispenser.

In some embodiments, the applicator or dispenser is a cloth, a wipe, a sponge, a mop, a squirt bottle, a spray bottle, a pump bottle, a tube, an automatic induction hand sterilizer, a bottle or container comprising a dropper, bottle or container comprising a pour spout, or a canister.

In some embodiments, the spray bottle is a continuous spray bottle, a propellant-free continuous spray bottle, a flairosol sprayer, an aerosol sprayer, or a mist spray bottle.

In some embodiments, the composition is formulated for topical administration.

In some embodiments, the composition comprises about 40% by weight of the ionic liquid.

In an aspect, there is provided a kit comprising the pharmaceutical gel composition comprising:
(i) an ionic liquid having a cationic component and an anionic component; and
(ii) at least one pharmaceutically carrier,
wherein the composition is non-irritating to skin, and wherein the composition comprises about 35% to about 45% by weight of the ionic liquid;
and a dispenser or applicator.

In an aspect, there is provided a method of inhibiting a member of the Kallikrein (KLK) serine protease family in a subject in need thereof, comprising administering to the subject a therapeutically effective amount of a pharmaceutical gel composition comprising:
(i) an ionic liquid having a cationic component and an anionic component; and
(ii) at least one pharmaceutically carrier,
wherein the composition is non-irritating to skin, and wherein the composition comprises about 35% to about 45% by weight of the ionic liquid.

In an aspect, there is provided a method of preventing, treating, and/or ameliorating at least one symptom of a KLK-mediated disease or disorder in a subject in need thereof, comprising administering to the subject a therapeutically effective amount of a pharmaceutical gel composition comprising:
(i) an ionic liquid having a cationic component and an anionic component; and
(ii) at least one pharmaceutically carrier,
wherein the composition is non-irritating to skin, and wherein the composition comprises about 35% to about 45% by weight of the ionic liquid.

In an aspect, there is provided a method of preventing or treating a KLK-mediated disease or disorder in a subject in need thereof, comprising administering to the subject a therapeutically effective amount of a pharmaceutical gel composition comprising:
(i) an ionic liquid having a cationic component and an anionic component; and
(ii) at least one pharmaceutically carrier,
wherein the composition is non-irritating to skin, and wherein the composition comprises about 35% to about 45% by weight of the ionic liquid.

In some embodiments, the KLK is Kallikrein 5 (KLK 5).

In some embodiments, the KLK is KLK 5.

In some embodiments, the KLK-mediated disease or disorder is a skin disease or disorder.

In some embodiments, the KLK-mediated disease or disorder is an inflammatory or infectious skin disease or condition.

In some embodiments, the skin disease or disorder is a disease or disorder of the dermis or epidermis.

In some embodiments, the skin disease or disorder is an acneiform eruption, an autoinflammatory syndrome, chronic blistering, a dermal growth, a dermatitis, a drug eruption, an endocrine-related skin condition or disorder, epidermal nevi, epidermal neoplasm, epidermal cyst, an erythema, genodermatoses, an infection or infection-related disorder, a lichenoid eruption, a lymphoid-related disorder, a pruritic disorder or condition, psoriasis, a recalcitrant palmoplantar eruption, urticarial, angioedema, a vascular-related disorder or condition, noninfectious immunodeficiency-related, a wound or physical trauma to the skin, a parasitic infestation, parasitic sting, parasitic bite, or a papulosquamous hyperkeratotic disorder or condition.

In some embodiments, the infection is caused by viral growth, bacterial growth, fungal growth, growth of a mold, growth of protozoan, parasitic growth, or combinations thereof.

In some embodiments, the bacteria is a gram-negative bacteria or a gram-positive bacteria.

In some embodiments, the skin disease or disorder is dermatitis, acne, wound, rash, folliculitis, furunculosis, carbunculosis fungal infection, or rosacea.

In some embodiments, the at least one symptom is redness, lesions, pustules, pruritis, skin irritation, or combinations thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1C. Schematic illustration of mode of action of CAGE for rosacea. (A) Synthetic scheme of CAGE/CGB400 gel, deep eutectic mixture; (B) CGB400 upon topical application, extracts lipid from the lipid bilayer and penetrates deep into the skin layers; (C) CAGE neutralizes pathogens, modulates TLR2 signaling and inhibits KLK5 and thus generation of proinflammatory peptides.

FIGS. 2A-2D. Therapeutic activity and skin permeation of CGB400. (A) Broth microdilution MICs for choline bicarbonate, geranic acid and CAGE1:2 (****P<0.0001); (B) Schematic of KLK5 inhibition pathway (C) IC50 curve of CAGE; (D) Comparison of in-vitro skin quantification of choline and geranic acid in human cadaver skin following CGB400 gel application. Data are averages±SEM, statistics by one-way ANOVA with Tukey HSD post-test. ****P<0.0001.

FIGS. 3A-3C. GLP dermal toxicity of CGB400 in minipigs. (A) Dosing regimen in Gottingen minipig; Plasma concentrations of choline (B) and geranic acid (C) on Days 1 and 91 in comparison to control following dermal administration.

FIGS. 4A-4B. Efficacy in patient subjects. Overview of (A) number of patients in different groups at the two sites; (B) mean of papules/pustules in patients with “Redness with bumps/blemishes.”

FIGS. 5A-5C. CAGE characterization, NMR and HPLC analyses. (A, B) NMR spectra of geranic acid, and CAGE_{1:2} respectively; (C) HPLC method development for choline and geranic acid.

FIGS. 6A-6C. CAGE_{1:2} characterization, DSC, TGA and GVS analyses. (A) DSC spectra modulated thermal analysis of CGB400 gel; (B) TGA analysis of CGB400 gel measuring % wet weight of water loss; (C) GVS spectra indicating water uptake with respect to dry weight.

FIGS. 7A-7D. CAGE_{1:2} characterization, DSC thermal analysis. (A) Cooling cycle. Glass transition marked in Rev Heat Flow (˜−69 C). Small exotherm in total heat flow (˜−80 C); (B) Cooling cycle illustrating modulated heat flow, expanded at low temperature. Some signal distortion <˜−80° C. Exotherm in nonreversing heat flow ca. −80 C; (C) Heating cycle. Glass transition marked in Rev Heat Flow (˜−69 C). Slight endotherm near glass transition. Slight endotherm in total heat flow (˜−70 C) and more apparent in nonreversing heat flow near glass transition, possibly enthalpic relaxation; (D) Heating cycle illustrating modulated heat flow, expanded at low temperature. Change in modulated heat flow consistent with glass transition. Some signal distortion <˜−80° C.

FIGS. 8A-8C. CAGE_{1:2} characterization, FTIR analysis. FTIR spectra of choline (A), geranic acid (B), and CAGE_{1:2} (C).

FIGS. 9A-9B. Antimicrobial/anti-inflammatory effects of CGB400. (A) IC50 curve of Aprotinin (positive control); (B) OD 405 nm Reading of hKLK5 Inhibition by CAGE_{1:2}.

FIGS. 10A-10D. TK profiling in Göttingen Minipigs Plasma on Day 1 and 91. Maximum mean plasma concentrations (Cmax) of choline in (A) males; (B) females and geranic acid in (C) males; (D) females. For choline, there was a lack of dose response, however for geranic acid, Cmax increased dose dependently and in a more-than dose proportional manner. Data are averages±SEM, statistics by two-way ANOVA with Tukey HSD post-test. *P<0.05, **P<0.01, ***P<0.001, ****P<0.0001.

FIGS. 11A-11B. Effectiveness evaluations based on PGA. Quantification of redness per visits based on PGA in subjects with (A) Redness only; and (B) Redness with Bumps/Blemishes.

FIGS. 12A-12B. Effectiveness evaluations based on IGA and IGAR. Quantification of redness per visits based on (A) IGA in subjects with Bumps/Blemishes; (B) IGAR in all subjects.

FIG. 13. Antibacterial activity of CAGE_{1:2} and pure individual components against Propionibacterium acnes.

FIG. 14. Safety Data: Adverse event profile in Subjects WITH (n=27) and WITHOUT (n=25) Bumps/Blemishes.

### DETAILED DESCRIPTION OF THE DISCLOSURE

Described herein, in certain embodiments, are compositions and methods for treating a skin condition in an individual in need thereof. In some embodiments, the skin condition is rosacea. In some embodiments, the method comprises administering to a skin of the individual a composition comprising an ionic liquid and a pharmaceutically acceptable solvent. In some embodiments the ionic liquid comprises a cation and an anion. In some embodiments, the ionic liquid comprises a choline cation and a fatty acid anion. In some embodiments, the fatty acid anion is a geranic acid anion. In some embodiments, the cation has anti-inflammatory properties. In some embodiments, the anion has anti-microbial properties. In some embodiments, the pharmaceutically acceptable solvent is water, ethanol, diisopropyl adipate, polyethylene glycol (PEG), glycerin, propylene glycol, or a combination thereof. In some embodiments, the composition further comprises a gelling agent.

Disclosed herein, in certain embodiments, are methods for treating a disease or a condition related to rosacea in an individual in need thereof, comprising administering to a skin of the individual a composition comprising: (a) an ionic liquid comprising a choline cation and geranic acid anion; and (b) a pharmaceutically acceptable solvent. In some embodiments, the pharmaceutically acceptable solvent is selected from the group consisting of: water, ethanol, diisopropyl adipate, polyethylene glycol (PEG), glycerin, propylene glycol, and a combination thereof. In some embodiments, the composition further comprises a gelling agent. In some embodiments, the gelling agent is selected from the group consisting of: hydroxyethyl cellulose (HEC), hydroxypropyl cellulose (HPC), hydroxypropylmethyl cellulose (HPMC), and a combination thereof.

In some embodiments, the ionic liquid comprises the choline cation and geranic acid anion in a molar ratio of 1:1 or 1:2 of choline cation to geranic acid anion. In some embodiments, the ionic liquid comprises the choline cation and geranic acid anion in a molar ratio in a range of 1:1 to 1:4 of choline cation to geranic acid anion. In some embodiments, the ionic liquid comprises the choline cation and geranic acid anion in a molar ratio of 1:1, 1:2, 1:3, or 1:4 of choline cation to geranic acid anion. In some embodiments, the composition provides an increased antimicrobial action compared to an antimicrobial action of choline or an antimicrobial action of geranic acid. In some embodiments, the increased antimicrobial action is a 10 fold less concentration of the composition required for complete killing of a microbe relative to a concentration of choline or a concentration of geranic acid required for complete killing of the microbe. In some embodiments, the composition provides an increased skin permeation relative to a skin permeation of choline or a skin permeation of geranic acid. In some embodiments, the composition provides an increased conductivity relative to a conductivity of geranic acid and a decreased conductivity relative to a conductivity of choline. In some embodiments, the ionic liquid comprises a concentration of about 0.1% to 99% of the composition, and the pharmaceutically acceptable solvent comprises a concentration of about 1% to about 99.9% of the composition.

In some embodiments, the composition is formulated for transdermal administration. In some embodiments, the composition further comprises an additional therapeutic agent selected from the group consisting of: a small molecule drug, an antimicrobial agent, a protein, a peptide, an antibody, a nucleic acid, a chemotherapy agent, and a combination thereof. In some embodiments, the composition is formulated as a gel, lotion, cream, ointment, solution, or a patch. In some embodiments, erythema of the skin of the individual is reduced. In some embodiments, inflammation of the skin of the individual is reduced. In some embodiments, lesions on the skin of the individual are reduced.

In some embodiments, the composition further comprises a fragrance agent. In some embodiments, the fragrance agent is an acid or a terpene of a citrus fruit. In some embodiments, the citrus fruit is an orange, a grapefruit, a lime, or a lemon. In some embodiments, the terpene is D-limonene. In some embodiments, the acid is citric acid or a derivative thereof.

Disclosed herein, in certain embodiments, are methods for treating a skin condition in an individual in need thereof, comprising administering to a skin of the individual a composition comprising: (a) an ionic liquid comprising a choline cation and a geranic acid anion; and (b) a pharmaceutically acceptable solvent selected from the group consisting of: diisopropyl adipate, polyethylene glycol (PEG), glycerin, propylene glycol. Further disclosed herein, in certain embodiments, are methods for treating a skin condition in an individual in need thereof, comprising administering to a skin of the individual a composition comprising: (a) an ionic liquid comprising a choline cation and a geranic acid anion; (b) a pharmaceutically acceptable solvent selected from the group consisting of: water, ethanol, diisopropyl adipate, polyethylene glycol (PEG), glycerin, propylene glycol, and a combination thereof; and (c) a gelling agent.

In some embodiments, the skin condition is rosacea. In some embodiments, the skin condition is caused by a mite, bacteria, or a combination thereof. In some embodiments, the composition does not induce development of resistance in the mite or the bacteria. In some embodiments, erythema of the skin of the individual is reduced. In some embodiments, inflammation of the skin of the individual is reduced. In some embodiments, lesions on the skin of the individual are reduced. In some embodiments, redness on the skin is reduced.

In some embodiments, the ionic liquid comprises the choline cation and geranic acid anion in a molar ratio of 1:1 or 1:2 of choline cation to geranic acid anion. In some embodiments, the ionic liquid comprises the choline cation and geranic acid anion in a molar ratio in a range of 1:1 to 1:4 of choline cation to geranic acid anion. In some embodiments, the ionic liquid comprises the choline cation and geranic acid anion in a molar ratio of 1:1, 1:2, 1:3, or 1:4 of choline cation to geranic acid anion. In some embodiments, the composition provides an increased antimicrobial action compared to an antimicrobial action of choline or an antimicrobial action of geranic acid. In some embodiments, the increased antimicrobial action is a 10 fold less concentration of the composition required for complete killing of a microbe relative to a concentration of choline or a concentration of geranic acid required for complete killing of the microbe. In some embodiments, the composition provides an increased skin permeation relative to a skin permeation of choline or a skin permeation of geranic acid. In some embodiments, the composition provides an increased conductivity relative to a conductivity of geranic acid and a decreased conductivity relative to a conductivity of choline. In some embodiments, the ionic liquid comprises a concentration of about 0.1% to 99% of the composition, and the pharmaceutically acceptable solvent comprises a concentration of about 1% to about 99.9% of the composition.

In some embodiments, the composition is formulated for transdermal administration. In some embodiments, the composition further comprises an additional therapeutic agent selected from the group consisting of: a small molecule drug, an antimicrobial agent, a protein, a peptide, an antibody, a nucleic acid, a chemotherapy agent, and a combination thereof. In some embodiments, the composition is formulated as a gel, lotion, cream, ointment, solution, or a patch. In some embodiments, the gelling agent is selected from the group consisting of: hydroxyethyl cellulose (HEC), hydroxypropyl cellulose (HPC), hydroxypropylmethyl cellulose (HPMC), and a combination thereof.

Disclosed herein, in certain embodiments, are compositions comprising: (a) an ionic liquid comprising a choline cation and a geranic acid anion; and (b) a pharmaceutically acceptable solvent selected from the group consisting of: diisopropyl adipate, polyethylene glycol (PEG), glycerin, propylene glycol, and a combination thereof. Further disclosed herein, in certain embodiments, are compositions comprising: (a) an ionic liquid comprising a choline cation and a geranic acid anion; (b) a pharmaceutically acceptable solvent selected from the group consisting of: water, ethanol, diisopropyl adipate, polyethylene glycol (PEG), glycerin, propylene glycol, and a combination thereof; and (c) a gelling agent.

In some embodiments, the ionic liquid comprises the choline cation and geranic acid anion in a molar ratio of 1:1 or 1:2 of choline cation to geranic acid anion. In some embodiments, the ionic liquid comprises the choline cation and geranic acid anion in a molar ratio in a range of 1:1 to 1:4 of choline cation to geranic acid anion. In some embodiments, the ionic liquid comprises the choline cation and geranic acid anion in a molar ratio of 1:1, 1:2, 1:3, or 1:4 of choline cation to geranic acid anion. In some embodiments, the composition provides an increased antimicrobial action compared to an antimicrobial action of choline or an antimicrobial action of geranic acid. In some embodiments, the increased antimicrobial action is a 10 fold less concentration of the composition required for complete killing of a microbe relative to a concentration of choline or a concentration of geranic acid required for complete killing of the microbe. In some embodiments, the composition provides an increased skin permeation relative to a skin permeation of choline or a skin permeation of geranic acid. In some embodiments, the composition provides an increased conductivity relative to a conductivity of geranic acid and a decreased conductivity relative to a conductivity of choline. In some embodiments, the ionic liquid comprises a concentration of about 0.1% to 99% of the composition, and the pharmaceutically acceptable solvent has a concentration of about 1% to about 99.9% of the composition.

In some embodiments, the composition is formulated for transdermal administration. In some embodiments, the composition further comprises an additional therapeutic agent selected from the group consisting of: a small molecule drug, an antimicrobial agent, a protein, a peptide, an antibody, a nucleic acid, a chemotherapy agent, and a combination thereof. In some embodiments, the composition is formulated as a gel, lotion, cream, ointment, solution, or a patch. In some embodiments, the gelling agent is selected from the group consisting of: hydroxyethyl cellulose (HEC), hydroxypropyl cellulose (HPC), hydroxypropylmethyl cellulose (HPMC), and a combination thereof.

In some embodiments, the composition comprises 20% to 60% of an ionic liquid comprising a choline cation and a geranic acid anion, 5% to 20% propylene glycol, and a remaining balance of water. In some embodiments, the composition comprises 30% to 50% of the ionic liquid. In some embodiments, the composition comprises a molar ratio of the choline cation and geranic acid anion of 1:2. In some embodiments, the composition comprises 10% to 15% propylene glycol. In some embodiments, the composition further comprises 0.5% to 5% hydroxyethyl cellulose. In some embodiments, the composition further comprises 0.5% to 5% D-limonene. In some embodiments, the composition is formulated as a gel. In some embodiments, the composition is formulated for topical administration. In some embodiments, the composition is formulated for twice daily administration.

### I. Definitions

The terminology used herein is for the purpose of describing particular cases only and is not intended to be limiting. The below terms are discussed to illustrate meanings of the terms as used in this specification, in addition to the understanding of these terms by those of skill in the art. As used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Certain ranges are presented herein with numerical values being preceded by the term “about.” The term “about” is used herein to provide literal support for the exact number that it precedes, as well as a number that is near to or approximately the number (±10%) that the term precedes. In determining whether a number is near to or approximately a specifically recited number, the near or approximating un-recited number may be a number which, in the context in which it is presented, provides the substantial equivalent of the specifically recited number. Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions described herein are. The upper and lower limits of these smaller ranges may independently be included in the smaller ranges and are also encompassed within the methods and compositions described herein, subject to any specifically excluded limit in the stated range. Where the stated range includes one or both of the limits, ranges excluding either or both of those included limits are also included in the methods and compositions described herein.

The terms “individual,” “patient,” or “subject” are used interchangeably. None of the terms require or are limited to situation characterized by the supervision (e.g., constant or intermittent) of a health care worker (e.g. a doctor, a registered nurse, a nurse practitioner, a physician's assistant, an orderly, or a hospice worker). Further, these terms refer to human or animal subjects.

“Treating” or “treatment” refers to both therapeutic treatment and prophylactic or preventative measures, wherein the object is to prevent or slow down (lessen) a targeted pathologic condition or disorder. Those in need of treatment include those already with the disorder, as well as those prone to have the disorder, or those in whom the disorder is to be prevented. For example, a subject or mammal is successfully “treated” for rosacea, if, after receiving a therapeutic amount of a composition according to the methods of the present disclosure, the subject shows observable and/or measurable reduction in or absence of one or more of the following: reduction in the erythema; reduction in the appearance of red veins; papules, and pustules.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of the compound, age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

Unless defined otherwise, all technical and scientific terms used herein have the same meaning as commonly understood by one of ordinary skill in the art to which the methods and compositions described herein belong. Although any methods and materials similar or equivalent to those described herein can also be used in the practice or testing of the methods and compositions described herein, representative illustrative methods and materials are now described.

### II. Ionic Liquid Compositions

In an aspect, there is provided a pharmaceutical gel composition, comprising:
(i) an ionic liquid having a cationic component and an anionic component; and
(ii) at least one pharmaceutically carrier, wherein the composition is non-irritating to skin, and wherein the composition comprises about 25% to about 45% by weight of the ionic liquid.

In an aspect, there is provided a pharmaceutical gel composition, comprising:
(i) an ionic liquid having a cationic component and an anionic component; and
(ii) at least one pharmaceutically carrier, wherein the composition is non-irritating to skin, and wherein the composition comprises about 35% to about 45% by weight of the ionic liquid.

In another aspect, there is provided a pharmaceutical gel composition, comprising:
(i) a deep eutectic solvent having a cationic component and an anionic component; and
(ii) at least one pharmaceutically carrier,
wherein the composition is non-irritating to skin, and wherein the deep eutectic solvent has a melting point lower than the melting points of the cationic component and anionic component individually.

In some embodiments, at least one of the anionic component and cationic component is irritating to the skin when applied in the absence of the other component.

In some embodiments, the anionic component is bistriflimide, a geranate, an oleate, a hexanoate, dodecyldimethyl ammonia propane sulfonate, N-lauryl sarcosinate, or a geraniolate.

In some embodiments, the cationic component is benzyl pyridinium, benzyl dimethyl dodecyl ammonium, a choline cation, phosphonium, benzethonium, or a phosphonium.

In some embodiments, the phosphonium is a tetraalkyl phosphonium of structural Formula (I): PR₄, wherein R is a substituted or unsubstituted alkyl group.

In some embodiments, the cationic component is a choline cation.

In some embodiments, the anionic component is a geranate anion.

In some embodiments, the cationic component and the anionic component are in a molar ratio ranging from about 1:1 to about 1:2 (cationic component to anionic component).

In some embodiments, the cationic component and the anionic component are in a molar ratio of about 1:2 (cationic component to anionic component).

In some embodiments, the composition comprises from about 1.0% to about 90% of choline geranate by weight.

In some embodiments, the composition comprises from about 20% to about 60% of choline geranate by weight.

In some embodiments, the composition comprises from about 35% to about 45% of choline geranate by weight.

In some embodiments, the composition comprises about 40% of choline geranate by weight.

In some embodiments, the composition further comprises a pH adjuster, skin conditioner, dye, fragrance, gelling agent, humectant, emollient, antioxidant, preservative, solvent, co-solvent, or combinations thereof.

In some embodiments, the fragrance is D-limonene.

In some embodiments, the fragrance is present in the composition an amount of from about 0.05% to about 5% by weight.

In some embodiments, the fragrance is present in the composition an amount of from about 0.2% to about 1% by weight.

In some embodiments, the gelling agent is hydroxyethyl cellulose.

In some embodiments, the gelling agent is present in the composition an amount of from about 0.05% to about 5% by weight.

In some embodiments, the gelling agent is present in the composition an amount of from about 0.3% to about 1.5% by weight.

In some embodiments, the humectant is glycerin.

In some embodiments, the humectant is present in the composition an amount of from about 0.5% to about 5% by weight.

In some embodiments, the humectant is present in the composition an amount of from about 1% to about 2% by weight.

In some embodiments, the composition further comprises emollients.

In some embodiments, the composition further comprises propylene glycol.

The propylene glycol is present in the composition an amount of from about 5% to about 20% by weight.

In some embodiments, the composition further comprises water.

In some embodiments, the composition has a viscosity of from about 5,000 cP to about 25,000 cP.

In some embodiments, the composition has a pH of from about 6.0 to about 8.0.

In some embodiments, the composition is stable for up to at least about 6 months at a temperature of about 40° C.

In some embodiments, the composition is stable for up at least about 5 days at 80° C.

In some embodiments, the composition is stable to UV-A light for at least about 3 days.

In some embodiments, the composition is placed on or in an applicator or dispenser.

In some embodiments, the applicator or dispenser is a cloth, a wipe, a sponge, a mop, a squirt bottle, a spray bottle, a pump bottle, a tube, an automatic induction hand sterilizer, a bottle or container comprising a dropper, bottle or container comprising a pour spout, or a canister.

In some embodiments, the spray bottle is a continuous spray bottle, a propellant-free continuous spray bottle, a flairosol sprayer, an aerosol sprayer, or a mist spray bottle.

In some embodiments, the composition is formulated for topical administration.

In some embodiments, the composition comprises about 40% by weight of the ionic liquid.

Described herein, in certain embodiments, are compositions comprising an ionic liquid comprising a choline cation and a fatty acid anion. In some embodiments, the composition further comprises a pharmaceutically acceptable solvent. In some embodiments, the fatty acid is myristoleic acid, palmitoleic acid, sapienic acid, oleic acid, elaidic acid, geranic acid, vaccenic acid, linoleic acid, linoelaidic acid, α-linolenic acid, arachidonic acid, eicosapentaenoic acid, erucic acid, docosahexaenoic acid, propionic acid, butyric acid, valeric acid, hexanoic acid, enanthic acid, caprylic acid, pelargonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid. In some embodiments, the fatty acid is geranic acid. In some embodiments, the fatty acid comprises 9 to 14 carbons. In some embodiments, the ionic liquid is liquid at room temperature. In some embodiments, the ionic liquid is liquid below 100° C.

In some embodiments, the ionic liquid is a deep eutectic solvent (DES). In some embodiments, a DES comprises excess carboxylate which precludes 1:1 ion pairing. In some embodiments, a DES further comprises a hydrogen-bond donor. In some embodiments, the hydrogen-bond donor is urea or citric acid. In some embodiments, the solvent properties of a DES are adjusted by changing the hydrogen-bond donor. In some embodiments, the ammonium salt of a DES interacts with a hydrogen-bond donor. In some embodiments, the DES has a melting point lower than either of the individual components (e.g. fatty acid and choline).

In some embodiments, the ionic liquid comprises a molar ratio of a choline cation to a fatty acid anion of 1:0.5 to 1:10. In some embodiments, the molar ratio of the choline cation to the fatty acid anion is about 1:0.5, 1:0.6, 1:0.7, 1:0.8, 1:0.9, 1:1.0; 1:1.1, 1:1.2, 1:1.3, 1:1.4, 1:1.5, 1:1.6, 1:1.7, 1:1.8, 1:1.9, 1:2.0, 1:2.1, 1:2.2, 1:2.3, 1:2.4, 1:2.5, 1:2.6, 1:2.7, 1:2.8, 1:2.9, 1:3.0, 1:3.1, 1:3.2, 1:3.3, 1:3.4, 1:3.5, 1:3.6, 1:3.7, 1:3.8, 1:3.9, 1:4.0, 1:4.1, 1:4.2, 1:4.3, 1:4.4, 1:4.5, 1:4.6, 1:4.7, 1:4.8, 1:4.9, 1:5.0, 1:5.1, 1:5.2, 1:5.3, 1:5.4, 1:5.5, 1:5.6, 1:5.7, 1:5.8, 1:5.9, 1:6.0, 1:6.1, 1:6.2, 1:6.3, 1:6.4, 1:6.5, 1:6.6, 1:6.7, 1:6.8, 1:6.9, 1:7.0, 1:7.1, 1:7.2, 1:7.3, 1:7.4, 1:7.5, 1:7.6, 1:7.7, 1:7.8, 1:7.9, 1:8.0, 1:8.1, 1:8.2, 1:8.3, 1:8.4, 1:8.5, 1:8.6, 1:8.7, 1:8.8, 1:8.9, 1:9.0, 1:9.1, 1:9.2, 1:9.3, 1:9.4, 1:9.5, 1:9.6, 1:9.7, 1:9.8, 1:9.9, or about 1:10. In some embodiments, the molar ratio of the choline cation to the fatty acid anion is about 1:1.1, 1:1.2, 1:1.3, 1:1.4, 1:1.5, 1:1.6, 1:1.7, 1:1.8, 1:1.9, or 1:2.0.

In some embodiments, the choline cation and fatty acid anion are in a molar ratio in the ionic liquid. In some embodiments, the choline cation and fatty acid anion are in a molar ratio of 1:1. In some embodiments, the term Composition B is used herein to refer to a composition or an ionic liquid comprising a 1:1 molar ratio of choline cation to geranic acid anion. In some embodiments, Composition B does not comprise water.

In other embodiments, the choline cation and fatty acid anion are in a molar ratio of 1:2. In some embodiments, the term Composition A is used herein to refer to a composition or an ionic liquid comprising a 1:2 molar ratio of choline cation to geranic acid anion. In some embodiments, Composition A does not comprise water.

In some embodiments, the chemical structure of choline is:

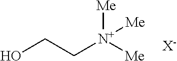

wherein X⁻ is a pharmaceutically acceptable anion.

In some embodiments, term choline refers to the class of quaternary ammonium salts containing the N,N,N-trimethylethanolammonium cation. In some embodiments, the X⁻ on the right of the structure of choline denotes a pharmaceutically acceptable anion. In some embodiments the X⁻ is bicarbonate, carbonate, acetate, citrate, tartarate, bitartarate, lactate, chloride, bromide, or iodide. In some embodiments, the X⁻ is bicarbonate. In some embodiments, the choline is an anti-inflammatory agent.

In some embodiments, choline is in the form of a pharmaceutically acceptable salt. The type of pharmaceutical acceptable salts, include, but are not limited to acid addition salts, formed by reacting the free base form of the compound with a pharmaceutically acceptable: inorganic acid such as hydrochloric acid, hydrobromic acid, sulfuric acid, nitric acid, phosphoric acid, metaphosphoric acid, and the like; or with an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

In some embodiments, the chemical structure of geranic acid, or 3,7-dimethyl-2,6-octadienoic acid, is:

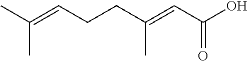

In some embodiments, geranic acid is in the form of a pharmaceutically acceptable salt. The type of pharmaceutical acceptable salts, include, but are not limited to salts formed when an acidic proton present in the parent compound either is replaced by a metal ion, e.g., an alkali metal ion (e.g. lithium, sodium, potassium), an alkaline earth ion (e.g., magnesium, or calcium), or an aluminum ion; or coordinates with an organic base. Examples of acceptable organic bases include, but are not limited to, ethanolamine, diethanolamine, triethanolamine, tromethamine, and N-methylglucamine. Examples of acceptable inorganic bases include, but are not limited to, aluminum hydroxide, calcium hydroxide, potassium hydroxide, sodium carbonate, and sodium hydroxide.

In some embodiments, the choline and the fatty acid are synthesized using any suitable standard synthetic reactions. In some embodiments, the reactions are employed in a linear sequence to provide the compounds or they may be used to synthesize fragments which are subsequently joined by any suitable method. In some embodiments, the starting material used for the synthesis of choline or fatty acid is synthesized or are obtained from commercial sources.

In some embodiments, geranic acid is purified from the commercially available technical grade (Sigma-Aldrich, St. Louis, Mo.) by repeated (5-7×) recrystallization from a solution of 70 wt % geranic acid/30 wt % acetone at −70° C. In some embodiments, purity of the geranic acid is assessed by ¹H NMR spectroscopy and conductivity measurements. In some embodiments, the term geranic acid refers to a geranic acid or a salt thereof. In some embodiments, the geranic acid is an anti-microbial agent.

In some embodiments, the pharmaceutically acceptable solvent is water, ethanol, diisopropyl adipate, polyethylene glycol (PEG), glycerin, propylene glycol, a short chain fatty acid, a fatty acid ester, or a combination thereof. In some embodiments, the pharmaceutically acceptable solvent is a liquid alcohol, liquid glycol, liquid polyalkalene glycol, liquid ester, liquid amine, liquid protein hydrolysate, liquid alkalated protein hydrolysate, liquid lanolin, lanolin derivative, or water. In some embodiments, the pharmaceutically acceptable solvent is diisopropyl adipate. In some embodiments, the composition is miscible with the pharmaceutically acceptable solvent. In some embodiments, at least one of the individual components of the composition is not miscible with pharmaceutically acceptable solvent. In some embodiments, the composition is miscible with diisopropyl adipate. In some embodiments, at least one of the individual components of the composition is not miscible with diisopropyl adipate. In some embodiments, the water is deionized water or Milli-Q® water. In some embodiments, the composition does not comprise a preservative. Examples of preservatives include, but are not limited to, a paraben or a phenoxyethanol.

In some embodiments, the composition comprises an increased antimicrobial action compared to an antimicrobial action of choline or an antimicrobial action of the fatty acid. In some embodiments, the increased antimicrobial action is a 10-fold less concentration of the composition required for complete killing of a microbe relative to a concentration of choline or a concentration of the fatty acid required for complete killing of the microbe.

In some embodiments, the composition comprises an increased skin permeation (or permeability) relative to a skin permeation of choline or a skin permeation of the fatty acid. In some embodiments, the composition increases skin permeation by disrupting the stratum corneum lipids, interacting with the intercellular proteins, improving portioning of the drug into the lipid layers, or a combination thereof. In some embodiments, the composition penetrates into the epidermis and dermis. In some embodiments, the composition achieves an Effect Site Concentration (Cₑₛ) in the dermis greater than the minimal inhibitory concentration (MIC) of the anti-microbial agent. In some embodiments, the anti-microbial agent is the fatty acid anion.

In some embodiments, the composition enhances delivery of small molecules, large molecules, or a combination thereof, through the skin. In some embodiments, small molecules have a molecular weight of less than 500 Da. In some embodiments, large molecules have a molecular weight of up to 150 kDa.

In some embodiments, the composition has decreased skin irritation relative to a skin irritation of choline or a skin irritation of the fatty acid. In some embodiments, the composition exhibits minimal cytotoxicity relative to a cytotoxicity of choline or a cytotoxicity of the fatty acid. In some embodiments, the composition comprises an increased conductivity relative to a conductivity of the fatty acid and a decreased conductivity relative to a conductivity of choline.

In some embodiments, the composition is clear. In some embodiments, the composition is turbid. In some embodiments, the composition is opaque. In some embodiments, the composition is yellow. In some embodiments, the composition is a colloidal system.

In some embodiments, the composition is formulated for transdermal administration. In some embodiments, the composition is formulated as a gel, lotion, cream, ointment, solution, or a patch. In some embodiments, the composition is formulated as a gel. In some embodiments, the patch is an adhesive-based patch or a reservoir-based patch. In some embodiments, the patch is a hypoallergenic patch.

In some embodiments, the composition further comprises a gelling agent, a viscosity modifying agent, or a combination thereof. In some embodiments, the gelling agent or the viscosity modifying agent is also a bulking agent.

Examples of gelling agents or viscosity modifying agents include, but are not limited to, as polyvinyl alcohol, polyethylene oxide, different poloxamers, carbopols, or celluloses such as ethyl cellulose, hydroxyl ethyl cellulose, hydroxyl propyl cellulose, hydroxyl propyl methyl cellulose, sulfoxides or similar compounds such as dimethylsulfoxide, dimethylsulfoxide, dimethylacetamide, dimethylformamide, pyrrolidones such as 2-pyrrolidone, N-methyl-2-pyrrolidone, 1-lauryl-2-pyrrolidone, alcohols such as ethanol, 1-octanol, 1-hexanol, 1-decanol, lauryl alcohol, linolenyl alcohol, glycols such as propylene glycol, butane-1,2-diol, polyethylene glycol 400, urea and derivatives urea, such as 1-dodecylurea, 1-dodecyl-3-methylurea,1-dodecyl-3-methylthiourea, azone and azone like molecules such as (laurocapram; 1-dodecylazacycloheptan-2-one), 1-alkyl- or 1-alkenylazacycloalkanones, enzymes acid phosphatase, calonase, papain Iminosulfuranes S,S-dimethyl-N-(5-nitro-2-pyridyl) iminosulfurane, S,S-dimethyl-N-(4-bromobenzoyl) iminosulfurane, cyclodextrins 2-hydroxypropyl-β-cyclodextrin, methylated-β-cyclodextrin, fatty acid esters such as cetyl lactate, butylacetate, isopropyl myristate Fatty acids alkanoic acids, oleic acid, lauric acid, capric acid, surfactants such as sorbitan monopalmitate, sorbitan trioleate, cetyl trimethyl ammonium bromide, sodium lauryl sulfate, terpenes such as limonene, nerolidol, farnesol, carvone, menthone, polymers such as β-D-glucopyranosyl-terminated oligodimethylsiloxanes, 1-alkyl-3-β-D-glucopyranosyl-1,1,3,3-tetramethyldisiloxanes Monoolein monoolein Oxazolidinones 4-decyloxazolidin-2-one, 3-acetyl-4-decyloxazolidin-2-one, carbomer, methyl cellulose, sodium carboxyl methyl cellulose, carrageenan, colloidal silicon dioxide, guar gum, gelatin, alginic acid, sodium alginate, and fumed silica. In some embodiments, the gelling agent is a hydroxyethyl cellulose (HEC), hydroxypropyl cellulose (HPC), hydroxypropylmethyl cellulose (HPMC), or a combination thereof. In some embodiments, the gelling agent is HPC.

In some embodiments, the combination of a gelling agent and a pharmaceutically acceptable solvent is referred to as a gel base. In some embodiments, a gel base is created prior to the addition of an ionic liquid to the gel base. In some embodiments, the ionic liquid is added into the gel base. In some embodiments, the gel base is added into the ionic liquid.

In some embodiments, the gel base comprises water and a gelling agent. In some embodiments, the gel base comprises diisopropyl adipate and a gelling agent. In some embodiments, the gel base comprises PEG400 and a gelling agent. In some embodiments, the gel base comprises propylene glycol and a gelling agent. In some embodiments, the gelling agent is HEC, HPC, or HPMC. In some embodiments, the gel base comprises ethanol and a gelling agent. In some embodiments, the gel base further comprises glycerin, propylene glycol, ethanol, or a combination thereof.

In one example, the gel base comprises diisopropyl adipate, ethanol, glycerin, and HPC. In some embodiments, the gel base comprises 25% w/w of diisopropyl adipate, 43% w/w ethanol, 30% w/w glycerin, and 3% w/w HPC. In another example, the gel base comprises diisopropyl adipate, ethanol, propylene glycol, and HPC. In some embodiments, the gel base comprises 25% w/w of diisopropyl adipate, 13% w/w ethanol, 60% w/w propylene glycol, and 3% w/w HPC.

In some embodiments, a composition comprises a bulking agent with a concentration from 1 to 10%. In some embodiments, a composition comprises a gelling agent with a concentration from 1 to 10%.

In some embodiments, the composition comprises an additional therapeutic agent selected from the group consisting of: a small molecule drug, an antimicrobial agent, a protein, a peptide, an antibody, a nucleic acid, a chemotherapy agent, and a combination thereof.

In some embodiments, the small molecule drug is a beta blocker, a loop diuretic, crotamiton, a retinoid, oxymetazoline hydrochloride, brimonidine, benzoyl peroxide, or a Janus kinase (JAK) inhibitor. In some embodiments, the beta blocker is propranolol, sotalol, atenolol, metoprolol, bisoprolol, carvedilol, nebivolol, or labetalol. In some embodiments, the loop diuretic is furosemide, bumetanide, or torsemide. In some embodiments, the small molecule drug is a vasodilator. In some embodiments, the retinoid is isotretinoin or adapalene. In some embodiments, the JAK inhibitor is tofacitinib, ocalcitinib, or ruxolitinib. In some embodiments, the small molecule drug is a prostacyclin analog. In some embodiments, the prostacyclin analog is treprostinil, epoprostenol, or iloprost. In some embodiments, the protein is insulin or albumin. In some embodiments, the composition comprises about 3.5 mg/mL of insulin. In some embodiments, the peptide is a dekapeptide. In some embodiments, the dekapeptide stimulates matrix regeneration, modulates melanin synthesis, stimulates lipolysis, deregulates cytokine release, or a combination thereof. In some embodiments, the chemotherapy agent is paclitaxel. In some embodiments, the composition comprises about 400 mg/mL of paclitaxel. In some embodiments, the nucleic acid is a small interfering RNA (siRNA) or a microRNA (miRNA). In some embodiments, the antimicrobial agent is a benzalkonium chloride, benzyl benzoate, sodium sulfacetamide, metronidazole, diaminodiphenyl sulfone (DDS; dapsone), permethrin, ivermectin, erythromycin, clindamycin, or azelaic acid. In some embodiments, the antimicrobial agent is an anti-acaride, anti-bacterial, anti-viral, anti-yeast, or anti-fungal agent. In some embodiments, the additional therapeutic agent is tea tree oil.

In some embodiments, the additional therapeutic agent is delivered into systemic circulation. In some embodiments, the additional therapeutic agent has low solubility.

In some embodiments, the composition further comprises a non-ionic surfactant. In some embodiments, the non-ionic surfactant is poloxamer or polysorbate 80. In some embodiments, the poloxamer is a Pluronic®, Kolliphor®, or Synperonic®. In some embodiments, the non-ionic surfactant comprises a concentration in the composition ranging from 0.1 to 20%.

In some embodiments, the composition further comprises an inactive ingredient. In some embodiments, the inactive ingredient enhances long-term shelf storage or target area absorption. In some embodiments, the inactive ingredient is an emollient/stiffening agents/ointment, an emulsifying agent/solubilizing agent, a humectant, a preservative, a permeation enhancer, a chelating agent, an antioxidant, vehicles/solvents, pH adjusting agents, or a combination thereof.

Example of emollients/stiffening agents/ointments include, but are not limited to, carnauba wax, cetyl alcohol, cetostearyl alcohol, cetyl ester wax, emulsifying wax, hydrous lanolin, lanolin, lanolin alcohols, microcrystalline wax, paraffin, petrolatum, polyethylene glycol and polymers thereof, stearic acid, stearyl alcohol, white wax, and yellow wax. Examples of emulsifying agents/solubilizing agents include, but are not limited to, glyceryl monostearate, glyceryl monooleate, glyceryl isostearate, polysorbate 20, polysorbate 80, polysorbate 60, poloxamer, emulsifying wax, sorbitan monostearate, sorbitan monooleate, sodium lauryl sulfate, propylene glycol monostearate, diethylene glycol monoethyl ether, and docusate sodium. Example of humectants include, but are not limited to, glycerin, propylene glycol, polyethylene glycol, sorbitol solution, and 1,2,6-hexanetriol. Examples of preservatives include, but are not limited to, benzoic acid, propyl paraben, methyl paraben, imidurea, sorbic acid, potassium sorbate, benzalkonium chloride, phenyl mercuric acetate, chlorobutanol, and phenoxyethanol. Examples of permeation enhances include, but are not limited to, propylene glycol, ethanol, isopropyl alcohol, oleic acid, and polyethylene glycol. Examples of chelating agents include, but are not limited to, ethylene diamine tetraacetate. Examples of antioxidants include, but are not limited to butylated hydroxyanisole and butylated hydroxytoluene. Examples of vehicles/solvents include, but are not limited to purified water, hexylene glycol, propylene glycol, oleyl alcohol, propylene carbonate, mineral oil, ethanol, diisopropyl adipate, polyethylene glycol (PEG), and glycerin. Examples of pH adjusting agents include, but are not limited to, acids such as acetic, boric, citric, lactic, phosphoric and hydrochloric acids; and bases such as sodium hydroxide, sodium phosphate, sodium borate, sodium citrate, sodium acetate, sodium bicarbonate, sodium lactate, ammonium chloride, and tris-hydroxymethylaminomethane. In some embodiments, the composition further comprises trolamine.

In some embodiments, the inactive ingredient is an acrylate or polymer thereof, methacrylate or polymer thereof, cellulose polymer, hydroxyethyl cellulose or polymer thereof, poly-lactylate polymer, polyvinyl pyrrolidone polymer, ethylenevinylacetate copolymer, short, medium and long chain fatty acid molecules or analog thereof, isopropryl myristate, polyethylene terephthalate, vitamin C, vitamin C analog or ester, vitamin E, vitamin E analog, vitamin E polymeric compound, d-α-tocopheryl polyethylene glycol 1000 succinate (vitamin E TPGS), or silicone.

In some embodiments, the inactive ingredient comprises dual or multiple functionalities. For example, in one embodiment, polyethylene glycol is an emollient, humectant, and a permeation enhancer.

In some embodiments, each component in a composition, such as the ionic liquid, the pharmaceutically acceptable solvent, and optionally other components, is described a percent (%) of the composition. In some embodiments, the % of the composition is a percent concentration volume/volume (v/v) or a percent concentration weight/volume (w/v).

In some embodiments, the composition comprises the ionic liquid in a concentration of about 0.1% to 99%. In some embodiments, the composition comprises the ionic liquid in a concentration of about 1% to 40%. In some embodiments, the composition comprises the ionic liquid in a concentration of about 1% to 20%. In some embodiments, the composition comprises the ionic liquid in a concentration of about 5% to 20%. In some embodiments, the composition comprises the ionic liquid in a concentration of about 5% to 40%. In some embodiments, the composition comprises the ionic liquid in a concentration of about 20% to 40%. In some embodiments, the composition comprises the ionic liquid in a concentration of about 20% to 60%. In some embodiments, the composition comprises the ionic liquid in a concentration of about 20% to 80%.

In some embodiments, the composition comprises the ionic liquid in a concentration of about 0.1% to 99%, and the pharmaceutically acceptable solvent in a concentration of about 1% to about 99.9%. In some embodiments, the composition comprises the ionic liquid in a concentration of about 1% to 40%, and the pharmaceutically acceptable solvent in a concentration of about 60% to about 99%. In some embodiments, the composition comprises the ionic liquid in a concentration of about 20% to 40%, and the pharmaceutically acceptable solvent in a concentration of about 80% to about 99%. In some embodiments, the composition comprises the ionic liquid in a concentration of about 20% and the pharmaceutically acceptable solvent in a concentration of about 80%. In some embodiments, the composition comprises the ionic liquid in a concentration of about 40% and the pharmaceutically acceptable solvent in a concentration of about 60%.

In some embodiments, the composition further comprises ethanol. In some embodiments, the concentration of ethanol in the composition is about 1%, 5%, 10%, 20%, 30%, 40%, or 50%.

In some embodiments, the composition comprises the ionic liquid in a concentration of about 20% to 40% and a gel base in a concentration of about 60% to 80%. In some embodiments, the composition comprises the ionic liquid in a concentration of 20% and a gel base in a concentration of 80%. In some embodiments, the composition comprises the ionic liquid in a concentration of about 20% to 40%, propylene glycol in a concentration of 20-50%, glycerin in a concentration of 10-20%, ethanol in a concentration of about 10-20%, and hydroxyl propyl cellulose in a concentration of less than 5%.

In some embodiments, the composition comprises propylene glycol. In some embodiments, the concentration of propylene glycol in the composition is about 1%, 5%, 10%, 20%, 30%, 40%, or 50%. In some embodiments, the concentration of propylene glycol in the composition is in a range of 1% to 40%, 5% to 20%, or 10% to 15%.

In some embodiments, the composition comprises hydroxyethyl cellulose. In some embodiments, the concentration of hydroxyethyl cellulose in the composition is about 0.1%, 0.5%, 1.0%, 2.0%, 3.0%, 4.0%, or 5.0%. In some embodiments, the concentration of hydroxyethyl cellulose in the composition is in a range of 0.5% to 5.0%, 0.75% to 2.0%, or 1.0% to 1.5%.

In some embodiments, the composition comprises a fragrance agent. In some embodiments, the fragrance agent comprises or is derived from essential oils, absolutes, resinoids, resins, concretes, or synthetic perfume components such as hydrocarbons, alcohols, aldehydes, ketones, ethers, acids, acetals, ketals and nitriles, including saturated and unsaturated compounds, aliphatic, carbocyclic and heterocyclic compounds, or precursors of any of the above. Exemplary fragrant agents include, but are not limited to, eucalyptus (Eucalyptus globulus or Eucalyptus citriadora), pine needles (Picca excelsa), Ho-leaves (Cinnamomum camphora hosch), peppermint (Mentha piperita), neem tree (Azadirachta excelsa), bay leaves (Laurus nobilis), litsea (Litsea cubeba), citronella (Cymbopogon nardus), elemi (Canarium luzonicum), petitgrain citronniers lemon (Citrus limonum), grapefruit (Citrus paradisi), fir tree (Abies alba pectinata), lavender (Lavandula officinalis), bergamotte (Citrus aurantium bergamia), and rosemary (Rosmarinus officinalis). In some embodiments, the fragrance agent is derived from a citrus fruit including but not limited to, oranges, lemons, grapefruit, and limes. In some embodiments, the fragrance agent is an acid or terpene derived from a citrus fruit. In some embodiments, the fragrance agent is citric acid or a citric acid derivative. In some embodiments, the fragrance agent is limonene.

In some embodiments, the composition comprises D-limonene. In some embodiments, the concentration of D-limonene in the composition is about 0.1%, 0.5%, 1.0%, 2.0%, 3.0%, 4.0%, or 5.0%. In some embodiments, the concentration of D-limonene in the composition is in a range of 0.5% to 5.0%, 0.75% to 2.0%, or 1.0% to 1.5%.

In some embodiments, the composition comprises the ionic liquid in a concentration of about 5% to 40% and a gel base comprising the pharmaceutically acceptable solvent in a concentration of about 60% to 95%. In some embodiments, the composition comprises the ionic liquid in a concentration of about 5% to 40%, and a gel base in a concentration of about 60% to 95%, wherein the gel base comprises diisopropyl adipate, propylene glycol, and a poloxamer. In some embodiments, the poloxamer is a Pluronic®.

In some embodiments, the composition comprises the ionic liquid in a concentration of about 1% to 50%, and the pharmaceutically acceptable solvent in a concentration of about 50% to 99%. In some embodiments, the composition comprises the ionic liquid in a concentration of about 1% to 50%, and water in a concentration of about 50% to 99%. In some embodiments, the water is deionized water or Milli-Q® water.

In some embodiments, the composition comprises the ionic liquid in a concentration of about 1% to 50%, a pharmaceutically acceptable solvent in a concentration of about 1% to 50%, and a gelling agent in a concentration of about 1 to 5%. In some embodiments, the composition comprises the ionic liquid in a concentration of about 1% to 50%, water in a concentration of about 1% to 50%, and HPC in a concentration of about 1 to 5%.

In some embodiments, the pharmaceutically acceptable solvent is diisopropyl adipate. In some embodiments, the composition comprises diisopropyl adipate in a concentration of about 20%. In some embodiments, the composition comprises the ionic liquid in a concentration of about 1% to 40%, and diisopropyl adipate in a concentration of about 60% to about 99%.

In some embodiments, the composition comprises a gel base in a concentration of about 50% to 90% of the composition. In some embodiments, the composition comprises a gel base in a concentration of about 50%, 60%, 70%, 80%, or 90% of the composition.

In some embodiments, preparing an ionic liquid comprising a choline cation and a fatty acid anion comprises: (a) mixing choline and a fatty acid in a solvent at room temperature in a predetermined ratio; and (b) removing the solvent in vacuo. In some embodiments, the fatty acid is geranic acid. In some embodiments, the solvent is water. In a particular embodiment, the water is deionized water. In some embodiments, removing the solvent comprises rotary evaporation. In some embodiments, removing the solvent comprises heating the ionic liquid, applying a vacuum to the ionic liquid, or a combination thereof. In some embodiments, preparing the ionic liquid further comprises drying the ionic liquid. In some embodiments, heating the ionic liquid comprises heating the ionic liquid to 60° C. In some embodiments, the heating is done for at least 10 minutes, 20 minutes, 30 minutes, 1 hour, 2 hours, 3 hours, 4 hours, 5 hours, 6 hours, 7 hours, 8 hours, 9 hours, 10 hours, 11 hours, 12 hours, 24 hours, 36 hours, 48 hours or 60 hours. In some embodiments, the vacuum is applied at −100 kPa. In some embodiments, the vacuum is applied for at least 10 minutes, 20 minutes, 30 minutes, 1 hour, 2 hours, 3 hours, 4 hours, 5 hours, 6 hours, 7 hours, 8 hours, 9 hours, 10 hours, 11 hours, 12 hours, 24 hours, 36 hours, 48 hours or 60 hours.

In some embodiments, the ionic liquid has had the solvent used in the ionic liquid preparation process removed. In some embodiments, the ionic liquid does not comprise water.

In some embodiments, choline is choline bicarbonate. In some embodiments, the choline is choline in an 80% wt solution of choline bicarbonate. In some embodiment, the predetermined ratio is a ratio of 1:1, 1:2, 1:3, or 1:4 of a choline cation:fatty acid anion. In one embodiment, the ratio is a molar ratio. In another embodiment, the ratio is ratio by weight.

In some embodiments, isolating the composition further comprises purifying the ionic liquid. In some embodiments, purifying the ionic liquid comprises using conventional techniques, including, but not limited to, filtration, distillation, crystallization, and chromatography. In some embodiments, preparing the ionic liquid further comprises isolating the purified ionic liquid.

The pre-clinical development and clinical validation of an ionic liquid/deep eutectic solvent CAGE for the treatment of rosacea is described herein.

CAGE_{1:2} was synthesized using a one-step salt metathesis reaction. Process development approaches were leveraged to overcome the limitations of laboratory scale CAGE_{1:2} synthesis. Industrially relevant, commercial reactor and processes enabled monitoring of reaction duration, CO₂ evolution and temperature for maintaining key quality attributes like pH, viscosity, color, odor and conductivity. The staple synthetic steps further rendered the manufacturing scale-up of CAGE_{1:2} and eventually CGB400 highly economical and straightforward. Furthermore, no additional preservatives were required in the final product, CGB400 topical gel owing to the inherent antimicrobial properties of CAGE_{1:2}. Methods for characterizing the product were developed and used to assess the finished product as well as its stability. The formulation exhibited excellent stability under stressed storage conditions.

Without wishing to be bound by theory, mechanistically, CAGE_{1:2} targets two key pathways know in rosacea. Specifically, higher densities of Staphylococcus epidermidis have been thought to be responsible for inflammation associated with rosacea. Further, activation of epidermal proteases, especially kallikrein (KLK-5) is also associated with rosacea and may further contribute to the inflammatory process. KLK-5 is a protease responsible for synthesis of antimicrobial peptides and its overexpression in the skin increases the local concentrations of the antimicrobial peptide, thus leading to overactivation of the local innate immune response which in turn causes cutaneous inflammation, vasodilation, and vascular proliferation. Activation of reactive oxygen species and hydroxyl radicals in the skin may also be a part of the inflammatory response, suggesting the need for agents modulating wider mechanistic and therapeutic targets.

CAGE_{1:2} exhibited efficacy against both targets. It exhibited strong activity against ten different clinical isolates of P. acnes. Previous studies have shown that CAGE_{1:2} penetrates into bacterial membrane and induces it disruption. Upon arriving near the bacterial membrane, choline interacts with the negatively charged bacterial membrane, thus inducing the geranic acid tail to enter the bacterial membrane leading to disruption. The mechanism by which CAGE_{1:2} inhibits KLK serine protease family, KLK5, is unclear. IC₅₀ of CAGE against KLK5 was found to be ˜30 mM. In vitro skin permeation studies in human skin indicated that the epidermal and dermal concentration after 24 hours far exceeded this value, thus confirming that the therapeutic concentration of CAGE_{1:2} can be achieved.

In vitro diffusion cell studies indicated that CAGE_{1:2} exhibited quick and extensive permeation into the epidermis and dermis. About 9.5% of the applied CAGE_{1:2} dose penetrated into the dermis within 24 hours, as determined by the amount of geranic acid recovered from the skin. Previous studies have established that CAGE_{1:2} contributes to lipid extraction from the stratum corneum lipid bilayers, which promotes skin's permeability to agents including CAGE itself. Simultaneous measurement of choline and geranic acid in the skin indicated that the ratio of choline:geranic acid in the epidermis was 1.18 after 24 hours. This is a surprising finding given that the lipophilicities of geranic acid and choline, as measured by octanol-water partition coefficients, differ by 7 orders of magnitude. This strongly suggests that choline and geranic acid maintain some interaction during their skin penetration. Furthermore, the higher concentration of choline in the skin and hence a higher molar ratio of choline:geranic acid (1.18) than that in the formulation (0.5) is likely due to the endogenous levels of choline.

GLP toxicity studies in minipigs indicated that long-term topical application of CGB400 for 91 days was generally well tolerated with no mortality and no macroscopic changes. Some dermal changes were observed but disappeared completely following 14-day recovery period which supports the safety profile of CGB400 gel. Furthermore, toxicokinetic profile indicated no significant increase in the measured blood choline concentrations from the existing levels. This could potentially have originated from the fact that dietary choline and/or natural choline metabolism dominated the blood concentrations. In contrast, the levels of geranic acid increased in the blood in a dose responsive manner. No systemic effects or organ toxicity of increased geranic acid concentration were seen.

CGB400 was well tolerated by patients, with most local side effects being mild to moderate. In an open-label 12-week cosmetic study, CGB400 demonstrated significant reduction in lesion counts (papules and pustules) on just visit 2 (2 weeks) with further reductions at subsequent study visits. At visit 5 (week 12), greatest reductions (˜71%) in lesions were reported, confirming the efficacy of CGB400 gel in improving rosacea symptoms. IGA responder rates and scores clearly demonstrated a marked improvement in symptoms with ˜70% of the subjects were deemed clear/almost clear. Likewise, IGAR scores strengthened until week 12, with ˜48% of clear/almost clear subjects. Furthermore, ˜73% subjects were deemed PGA responders after 12-week treatment. These findings confirm significant potential of CGB400 gel in the treatment of rosacea.

The studies reported here are a proof-of-concept assessment and demonstration of deep eutectic solvents/ionic liquids for treating skin condition, such as rosacea. While the studies reported here focus on rosacea, they provide guidance for many other potential products that use CAGE. In particular, the processes related to scale-up, characterization, stability, and GLP-toxicity could be common to many other products based on CAGE.

### III. Methods of Treating Skin Conditions

Disclosed herein, in certain embodiments, are methods for treating a skin condition in an individual in need thereof comprising administering to a skin of the individual a composition comprising: (a) an ionic liquid comprising a choline cation and a fatty acid anion; and (b) a pharmaceutically acceptable solvent. In some embodiments, the fatty acid anion is a geranic acid anion. In some embodiments, the individual is a mammal. In some embodiments, the mammal is a human.

In an aspect, there is provided a method of inhibiting a member of the Kallikrein (KLK) serine protease family in a subject in need thereof, comprising administering to the subject a therapeutically effective amount of a pharmaceutical gel composition comprising:
(i) an ionic liquid having a cationic component and an anionic component; and
(ii) at least one pharmaceutically carrier,
wherein the composition is non-irritating to skin, and wherein the composition comprises about 35% to about 45% by weight of the ionic liquid.

In an aspect, there is provided a method of preventing, treating, and/or ameliorating at least one symptom of a KLK-mediated disease or disorder in a subject in need thereof, comprising administering to the subject a therapeutically effective amount of a pharmaceutical gel composition comprising:
(i) an ionic liquid having a cationic component and an anionic component; and
(ii) at least one pharmaceutically carrier,
wherein the composition is non-irritating to skin, and wherein the composition comprises about 35% to about 45% by weight of the ionic liquid.

In an aspect, there is provided a method of preventing or treating a KLK-mediated disease or disorder in a subject in need thereof, comprising administering to the subject a therapeutically effective amount of a pharmaceutical gel composition comprising:
(i) an ionic liquid having a cationic component and an anionic component; and
(ii) at least one pharmaceutically carrier,
wherein the composition is non-irritating to skin, and wherein the composition comprises about 35% to about 45% by weight of the ionic liquid.

In some embodiments, the KLK is Kallikrein 5 (KLK 5).

In some embodiments, the KLK is KLK 5.

In some embodiments, the KLK-mediated disease or disorder is a skin disease or disorder.

In some embodiments, the KLK-mediated disease or disorder is an inflammatory or infectious skin disease or condition.

In some embodiments, the skin disease or disorder is a disease or disorder of the dermis or epidermis.

In some embodiments, the skin disease or disorder is an acneiform eruption, an autoinflammatory syndrome, chronic blistering, a dermal growth, a dermatitis, a drug eruption, an endocrine-related skin condition or disorder, epidermal nevi, epidermal neoplasm, epidermal cyst, an erythema, genodermatoses, an infection or infection-related disorder, a lichenoid eruption, a lymphoid-related disorder, a pruritic disorder or condition, psoriasis, a recalcitrant palmoplantar eruption, urticarial, angioedema, a vascular-related disorder or condition, noninfectious immunodeficiency-related, a wound or physical trauma to the skin, a parasitic infestation, parasitic sting, parasitic bite, or a papulosquamous hyperkeratotic disorder or condition.

In some embodiments, the infection is caused by viral growth, bacterial growth, fungal growth, growth of a mold, growth of protozoan, parasitic growth, or combinations thereof.

In some embodiments, the bacteria is a gram-negative bacteria or a gram-positive bacteria.

In some embodiments, the skin disease or disorder is dermatitis, acne, wound, rash, folliculitis, furunculosis, carbunculosis fungal infection, or rosacea.

In some embodiments, the at least one symptom is redness, lesions, pustules, pruritis, skin irritation, or combinations thereof.

In some embodiments, the skin condition is associated with infection. In some embodiments, the skin condition is associated with inflammation. In some embodiments, the skin condition is associated with inflammation and infection. In some embodiments, skin conditions associated with infection show symptoms of lesions, papules, pustules, or a combination thereof. In some embodiments, skin conditions associated with inflammation show symptoms of rashes, redness (erythema), persistent red veins, or a combination thereof.

In some embodiments, the skin condition is caused by a mite, bacteria, virus, yeast, or fungus. In some embodiments, treatment of the skin condition with the composition does not induce development of resistance in the mite, bacteria, virus, yeast, or fungus. In some embodiments, the mite is a Demodex mite. In some embodiments, the Demodex mite is Demodex folliculorum or Demodex brevis. In some embodiments, the bacteria is Bacillus oleronius or Staphylococcus epidermidis. In some embodiments, the bacteria is associated with the Demodex mite. In some embodiments, the rosacea is associated with proliferation of Demodex mites.

In some embodiments, the skin condition is rosacea. In some embodiments, the skin condition is impetigo, cold sore, wart, molluscum, onychomycosis, rosacea, or a combination thereof. In some embodiments, the skin condition is a skin condition caused by an overpopulation of Demodex mites, such as demodicosis.

In some embodiments, the skin condition causes erythema, inflammation, lesions, or a combination thereof on the skin of the individual. In some embodiments, a therapeutically effective amount of the composition is administered to the skin of the individual. In some embodiments, the composition is administered to an area of skin affected with the skin condition. In some embodiments, therapeutically effective amounts are determined by routine experimentation, including but not limited to a dose escalation clinical trial. In some embodiments, administration of the composition to the skin of the individual results in a reduction of erythema of the skin of the individual. In some embodiments, administration of the composition to the skin of the individual results in a reduction of inflammation of the skin of the individual. In some embodiments, inflammation is reduced by down regulating a cytokine. In some embodiment, the cytokine is a tumor necrosis factor alpha (TNFα), an interleukin, or a combination thereof. In some embodiments, administration of the composition to the skin of the individual results in a reduction of lesions on the skin of the individual.

In some embodiments, the composition is administered prophylactically to an individual susceptible or otherwise at risk of the skin condition.

In some embodiments, the amount of the composition administered to the individual and the length of treatment depends on the attributes of the individual including, but not limited to, state of health, weight, severity of the condition, previous therapy, and judgement of the treating physician. In some embodiments, the amount of the composition administered to the individual is determined by routine experimentation (e.g., a dose escalation clinical trial).

In some embodiments, the composition is applied to the skin of the individual once a day. In some embodiments, the composition is applied to the skin of the individual 1, 2, 3, 4, or 5 times a day. In some embodiments, the composition is applied to the skin of the individual 2 times a day. In some embodiments, the composition is applied to the skin of the individual 2 times a day, e.g., morning and evening. In some embodiments, the composition is applied to the skin of the individual every day, every other day, every three days, twice a week, once a week, or once a month. In some embodiments, the composition is applied to the skin of the individual once. In some embodiments, the composition is applied to the skin of the individual for a period of time of 1 week, 2 weeks, 3 weeks, 1 month, 2 months, or 3 months. In some embodiments, the composition is applied to the skin until the symptoms of the skin condition associated with infection are eliminated. In some embodiments, the composition is applied to the skin until the symptoms of the skin condition associated with inflammation are eliminated. In some embodiments, the composition is applied to the skin until the symptoms of the skin condition associated with infection are reduced. In some embodiments, the composition is applied to the skin until the symptoms of the skin condition associated with inflammation are reduced.

In some embodiments, compositions as described herein improve the symptoms of rosacea. In some embodiments, compositions as described herein decrease the number of inflammatory lesions. In some embodiments, compositions as described herein decrease the number of inflammatory lesions by 10%, 15%, 20%, 25%, 30%, 35%, 40%, 45%, 50%, 55%, 60%, 65%, 70%, 75%, 80%, 85%, 90%, 95%, or more than 95%. In some embodiments, compositions as described herein decrease the number of inflammatory lesions by at least or about 0.5×, 1.0×, 1.5×, 2.0×, 2.5×, 3.0×, 3.5×, 4.0×, 5.0×, 6.0×, 7.0×, 8.0×, 9.0×, 10×, or more than 10×. In some embodiments, compositions as described herein decrease the redness of the skin. In some embodiments, compositions as described herein decrease the redness of the skin by 10%, 15%, 20%, 25%, 30%, 35%, 40%, 45%, 50%, 55%, 60%, 65%, 70%, 75%, 80%, 85%, 90%, 95%, or more than 95%. In some embodiments, compositions as described herein decrease the redness of the skin by at least or about 0.5×, 1.0×, 1.5×, 2.0×, 2.5×, 3.0×, 3.5×, 4.0×, 5.0×, 6.0×, 7.0×, 8.0×, 9.0×, 10×, or more than 10×. In some embodiments, compositions as described herein improve skin complexion. In some embodiments, improved skin complexion comprises a reduction in redness, bumps, blemishes, or a combination thereof. In some embodiments, compositions as described herein improve skin complexion by 10%, 15%, 20%, 25%, 30%, 35%, 40%, 45%, 50%, 55%, 60%, 65%, 70%, 75%, 80%, 85%, 90%, 95%, or more than 95%. In some embodiments, compositions as described herein improve skin complexion by at least or about 0.5×, 1.0×, 1.5×, 2.0×, 2.5×, 3.0×, 3.5×, 4.0×, 5.0×, 6.0×, 7.0×, 8.0×, 9.0×, 10×, or more than 10×.

In some embodiments, compositions as described herein improve the symptoms of rosacea by a certain time. In some embodiments, compositions as described herein decrease the number of inflammatory lesions following at least or about 1 day, 2 days, 3 days, 4 days, 5 days, 6 days, 1 week, 2 weeks, 3 weeks, 1 month, 2 months, 3 months, or more than 3 months of administration. some embodiments, compositions as described herein decrease the number of inflammatory lesions by at least or about 10%, 15%, 20%, 25%, 30%, 35%, 40%, 45%, 50%, 55%, 60%, 65%, 70%, 75%, 80%, 85%, 90%, 95%, or more than 95% following at least or about 1 day, 2 days, 3 days, 4 days, 5 days, 6 days, 1 week, 2 weeks, 3 weeks, 1 month, 2 months, 3 months, or more than 3 months of administration. In some embodiments, compositions as described herein decrease the redness of the skin following at least or about 1 day, 2 days, 3 days, 4 days, 5 days, 6 days, 1 week, 2 weeks, 3 weeks, 1 month, 2 months, 3 months, or more than 3 months of administration. In some embodiments, compositions as described herein decrease the redness of the skin by at least or about 10%, 15%, 20%, 25%, 30%, 35%, 40%, 45%, 50%, 55%, 60%, 65%, 70%, 75%, 80%, 85%, 90%, 95%, or more than 95% following at least or about 1 day, 2 days, 3 days, 4 days, 5 days, 6 days, 1 week, 2 weeks, 3 weeks, 1 month, 2 months, 3 months, or more than 3 months of administration. In some embodiments, compositions as described herein decrease redness, bumps, blemishes, or a combination thereof following at least or about 1 day, 2 days, 3 days, 4 days, 5 days, 6 days, 1 week, 2 weeks, 3 weeks, 1 month, 2 months, 3 months, or more than 3 months of administration. Some embodiments, compositions as described herein decrease redness, bumps, blemishes, or a combination thereof by at least or about 10%, 15%, 20%, 25%, 30%, 35%, 40%, 45%, 50%, 55%, 60%, 65%, 70%, 75%, 80%, 85%, 90%, 95%, or more than 95% following at least or about 1 day, 2 days, 3 days, 4 days, 5 days, 6 days, 1 week, 2 weeks, 3 weeks, 1 month, 2 months, 3 months, or more than 3 months of administration.

Methods for determining improvements in the symptoms of rosacea, in some embodiments, comprise the Investigator's Global Assessment, the Investigator's Global Assessment of Redness, the Subject Global Assessment, or a combination thereof.

In some embodiments, administration of the composition is temporarily reduced or temporarily suspended for a certain length of time (i.e., a “drug holiday”). In some embodiments, the length of the drug holiday is between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. In some embodiments, the dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Rosacea is a common chronic inflammatory skin disorder affecting 415 million people worldwide and clinical manifestations include facial erythema, papulopustular lesions, phymatous changes, facial telangiectasias, intermittent flares of acute vasodilation (flushing). It is a phenotypically heterogenous disease with varying range of clinical manifestations and symptoms that makes the diagnosis and treatment challenging. While the underlying causes of rosacea remain poorly defined, therapies that target multiple contributing factors may offer advantages over current therapies.

A few topical products have been approved by the FDA for the treatment of rosacea such as including Mirvaso® (0.33% brimonidine) topical gel and Rhofade® (1% oxymetazoline hydrochloride) for persistent (non-transient) facial erythema of rosacea in adults. Additional products include Soolantra® (1% ivermectin cream), Finacea® (15% azelaic acid foam), and Metrogel® (1% metronidazole gel) which have gained interest for modulating immune responses and selective antiparasitic activity in rosacea pathophysiology. Mirvaso® and Rhofade® are vasoconstrictors and address transient redness. They have limited efficacy against persistent redness, inflammatory papules or pustules. Soolantra®, Finacea®, and Metrogel® have shown antimicrobial and anti-inflammatory effects in reducing the inflammatory papules and pustules associated with the disease, however their effect on persistent facial erythema has been limited. They are also limited by treatment cost and poor dermal permeation. Hence, the development of therapeutics that address these challenges can significantly improve the treatment and care of rosacea.

An ionic liquid/deep eutectic solvent comprised of choline and geranic acid (CAGE) has been shown to exhibit characteristics that make it a potential candidate for effective treatment of rosacea. Specifically, in vitro studies have shown that CAGE exhibits excellent antimicrobial activity against a broad variety of pathogens including Staphylococcus epidermidis which could be associated with pustules of rosacea. Further, CAGE has been shown to exhibit deep penetration into the skin, thus suggesting its ability to treat pathogens residing deep into the skin. Building on this scientific foundation, a formulation referred to as CGB400, comprising 40% w/w CAGE_{1:2} (choline:geranic acid ratio of 1:2) gel formulated in common pharmaceutical excipients is demonstrated herein. Scale-up, characterization, stability, mechanism of action, dosing, pre-clinical toxicology in minipigs, and human volunteer studies on safety and efficacy in treating rosacea are described herein. Without wishing to be limited by any particular theory, it is contemplated herein that these studies support the use of an ionic liquid-based approach for the treatment of dermatological conditions.

### IV. Non-Limiting Embodiments

Embodiment 1. A pharmaceutical gel composition, comprising:
   (i) an ionic liquid having a cationic component and an anionic component; and
   (ii) at least one pharmaceutically carrier,
   wherein the composition is non-irritating to skin, and wherein the composition comprises about 35% to about 45% by weight of the ionic liquid.
Embodiment 2. A pharmaceutical gel composition, comprising:
   (i) a deep eutectic solvent having a cationic component and an anionic component; and
   (ii) at least one pharmaceutically carrier,
   wherein the composition is non-irritating to skin, and wherein the deep eutectic solvent has a melting point lower than the melting points of the cationic component and anionic component individually.
Embodiment 3. The pharmaceutical gel composition of Embodiment 1 or 2, wherein at least one of the anionic component and cationic component is irritating to the skin when applied in the absence of the other component.
Embodiment 4. The pharmaceutical gel composition of any one of Embodiments 1-3, wherein the anionic component is bistriflimide, a geranate, an oleate, a hexanoate, dodecyldimethyl ammonia propane sulfonate, N-lauryl sarcosinate, or a geraniolate.
Embodiment 5. The pharmaceutical gel composition of any one of Embodiments 1-4, wherein the cationic component is benzyl pyridinium, benzyl dimethyl dodecyl ammonium, a choline cation, phosphonium, benzethonium, or a phosphonium.
Embodiment 6. The pharmaceutical gel composition of Embodiment 5, wherein the phosphonium is a tetraalkyl phosphonium of structural Formula (I): PR₄, wherein R is a substituted or unsubstituted alkyl group.
Embodiment 7. The pharmaceutical gel composition of any one of Embodiments 1-6, wherein the cationic component is a choline cation.
Embodiment 8. The pharmaceutical gel composition of any one of Embodiments 1-7, wherein the anionic component is a geranate anion
Embodiment 9. The pharmaceutical gel composition of any one of Embodiments 1-8, wherein the cationic component and the anionic component are in a molar ratio ranging from about 1:1 to about 1:2 (cationic component to anionic component).
Embodiment 10. The pharmaceutical gel composition of any one of Embodiments 1-9, wherein the cationic component and the anionic component are in a molar ratio of about 1:2 (cationic component to anionic component).
Embodiment 11. The pharmaceutical gel composition of any one of Embodiments 8-10, wherein the composition comprises from about 1.0% to about 90% of choline geranate by weight.
Embodiment 12. The pharmaceutical gel composition of any one of Embodiments 8-11, wherein the composition comprises from about 20% to about 60% of choline geranate by weight.
Embodiment 13. The pharmaceutical gel composition of any one of Embodiments 8-12, wherein the composition comprises from about 35% to about 45% of choline geranate by weight.
Embodiment 14. The pharmaceutical gel composition of any one of Embodiments 8-13, wherein the composition comprises about 40% of choline geranate by weight.
Embodiment 15. The pharmaceutical gel composition of any one of Embodiments 1-14, wherein the composition further comprises a pH adjuster, skin conditioner, dye, fragrance, gelling agent, humectant, emollient, antioxidant, preservative, solvent, co-solvent, or combinations thereof.
Embodiment 16. The pharmaceutical gel composition of Embodiment 15, wherein the fragrance is D-limonene.
Embodiment 17. The pharmaceutical gel composition of Embodiment 15 or 16, wherein the fragrance is present in the composition an amount of from about 0.05% to about 5% by weight.
Embodiment 18. The pharmaceutical gel composition of any one of Embodiments 15-17, wherein the fragrance is present in the composition an amount of from about 0.2% to about 1% by weight.
Embodiment 19. The pharmaceutical gel composition of any one of Embodiments 15-18, wherein the gelling agent is hydroxyethyl cellulose.
Embodiment 20. The pharmaceutical gel composition of any one of Embodiments 15-19, wherein the gelling agent is present in the composition an amount of from about 0.05% to about 5% by weight.
Embodiment 21. The pharmaceutical gel composition of any one of Embodiments 15-20, wherein the gelling agent is present in the composition an amount of from about 0.3% to about 1.5% by weight.
Embodiment 22. The pharmaceutical gel composition of any one of Embodiments 15-21, wherein the humectant is glycerin.
Embodiment 23. The pharmaceutical gel composition of any one of Embodiments 15-22, wherein the humectant is present in the composition an amount of from about 0.5% to about 5% by weight.
Embodiment 24. The pharmaceutical gel composition of any one of Embodiments 15-23, wherein the humectant is present in the composition an amount of from about 1% to about 2% by weight.
Embodiment 25. The pharmaceutical gel composition of any one of Embodiments 1-24, wherein the composition further comprises emollients.
Embodiment 26. The pharmaceutical gel composition of any one of Embodiments 1-25, wherein the composition further comprises propylene glycol.
Embodiment 27. The pharmaceutical gel composition of Embodiment 26, wherein the propylene glycol is present in the composition an amount of from about 5% to about 20% by weight.
Embodiment 28. The pharmaceutical gel composition of any one of Embodiments 1-27, wherein the composition further comprises water.
Embodiment 29. The pharmaceutical gel composition of any one of Embodiments 1-28, wherein the composition has a viscosity of from about 5,000 cP to about 25,000 cP.
Embodiment 30. The pharmaceutical gel composition of any one of Embodiments 1-29, wherein the composition has a pH of from about 6.0 to about 8.0.
Embodiment 31. The pharmaceutical gel composition of any one of Embodiments 1-30, wherein the composition is stable for up to at least about 6 months at a temperature of about 40° C.
Embodiment 32. The pharmaceutical gel composition of any one of Embodiments 1-31, wherein the composition is stable for up at least about 5 days at 80° C.
Embodiment 33. The pharmaceutical gel composition of any one of Embodiments 1-32, wherein the composition is stable to UV-A light for at least about 3 days.
Embodiment 34. The pharmaceutical gel composition of any one of Embodiments 1-33, wherein the composition is placed on or in an applicator or dispenser.
Embodiment 35. The pharmaceutical gel composition of Embodiment 34, wherein the applicator or dispenser is a cloth, a wipe, a sponge, a mop, a squirt bottle, a spray bottle, a pump bottle, a tube, an automatic induction hand sterilizer, a bottle or container comprising a dropper, bottle or container comprising a pour spout, or a canister.
Embodiment 36. The pharmaceutical gel composition of Embodiment 35, wherein the spray bottle is a continuous spray bottle, a propellant-free continuous spray bottle, a flairosol sprayer, an aerosol sprayer, or a mist spray bottle.
Embodiment 37. The pharmaceutical gel composition of any one of Embodiments 1-36, wherein the composition is formulated for topical administration.
Embodiment 38. The pharmaceutical gel composition of any one of Embodiments 1-37, wherein the composition comprises about 40% by weight of the ionic liquid.
Embodiment 39. A kit comprising the pharmaceutical gel composition of any one of Embodiments 1-38 and a dispenser or applicator.
Embodiment 40. A method of inhibiting a member of the Kallikrein (KLK) serine protease family in a subject in need thereof, comprising administering to the subject a therapeutically effective amount of the pharmaceutical gel composition of any one of Embodiments 1-39.
Embodiment 41. The method of Embodiment 40, wherein the KLK is Kallikrein 5 (KLK 5).
Embodiment 42. A method of preventing, treating, and/or ameliorating at least one symptom of a KLK-mediated disease or disorder in a subject in need thereof, comprising administering to the subject a therapeutically effective amount of the pharmaceutical gel composition of any one of Embodiments 1-39.
Embodiment 43. A method of preventing or treating a KLK-mediated disease or disorder in a subject in need thereof, comprising administering to the subject a therapeutically effective amount of the pharmaceutical gel composition of any one of Embodiments 1-39.
Embodiment 44. The method of Embodiment 42 or 43, wherein the KLK is KLK 5.
Embodiment 45. The method of any one of Embodiments 43-44, wherein the KLK-mediated disease or disorder is a skin disease or disorder.
Embodiment 46. The method of any one of Embodiments 43-45, wherein the KLK-mediated disease or disorder is an inflammatory or infectious skin disease or condition.
Embodiment 47. The method of any one of Embodiments 43-46, wherein the skin disease or disorder is a disease or disorder of the dermis or epidermis.
Embodiment 48. The method of any one of Embodiments 45-47, wherein the skin disease or disorder is an acneiform eruption, an autoinflammatory syndrome, chronic blistering, a dermal growth, a dermatitis, a drug eruption, an endocrine-related skin condition or disorder, epidermal nevi, epidermal neoplasm, epidermal cyst, an erythema, genodermatoses, an infection or infection-related disorder, a lichenoid eruption, a lymphoid-related disorder, a pruritic disorder or condition, psoriasis, a recalcitrant palmoplantar eruption, urticarial, angioedema, a vascular-related disorder or condition, noninfectious immunodeficiency-related, a wound or physical trauma to the skin, a parasitic infestation, parasitic sting, parasitic bite, or a papulosquamous hyperkeratotic disorder or condition.
Embodiment 49. The method of Embodiment 46 or 47, wherein the infection is caused by viral growth, bacterial growth, fungal growth, growth of a mold, growth of protozoan, parasitic growth, or combinations thereof.
Embodiment 50. The pharmaceutical gel composition of Embodiment 49, wherein the bacteria is a gram-negative bacteria or a gram-positive bacteria.
Embodiment 51. The method of any one of Embodiments 43-49, wherein the skin disease or disorder is dermatitis, acne, wound, rash, folliculitis, furunculosis, carbunculosis fungal infection, or rosacea.
Embodiment 52. The method of any one of Embodiments 43-51, wherein the at least one symptom is redness, lesions, pustules, pruritis, skin irritation, or combinations thereof.

### Non-Limiting Examples

Choline geranate deep eutectic solvent/ionic liquid (CAGE) has shown several desirable therapeutic properties including antimicrobial activity and ability to deliver drugs transdermally in research laboratories. In some embodiments, the clinical use of CAGE for the treatment of rosacea, a common chronic inflammatory skin disorder that affects the face is described herein. These results demonstrate the therapeutic potential of CGB400 for treating rosacea as well as insights into deep eutectic solvents, in particular CAGE, for dermatological applications.

### Example 1: Results

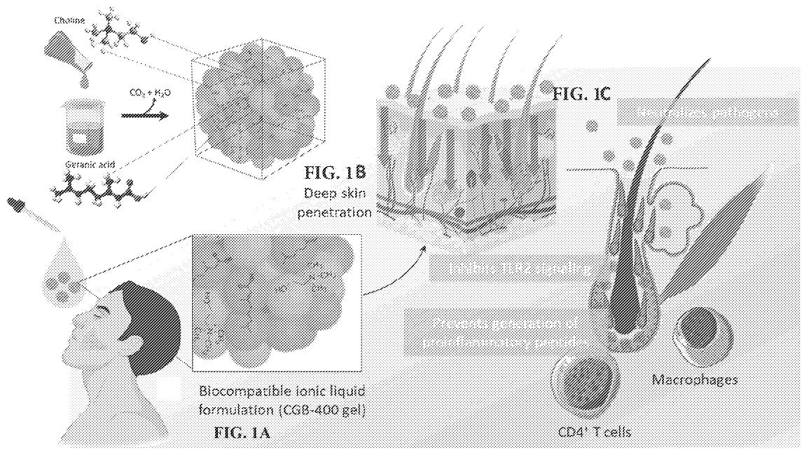

CGB400 synthesis and scale up. Scale-up of materials is often a complex process since some of the key functional aspects of the synthesis procedure may not scale proportionately. The active ingredient in CGB400, CAGE_{1:2}, was synthesized in a commercial reactor at a kilogram scale using the same process as that used at a smaller scale. Specifically, CAGE_{1:2} was synthesized by a one-step salt metathesis reaction between choline bicarbonate and geranic acid (FIG. 1A). No specific issues were observed during scale-up. CAGE_{1:2} is hygroscopic in nature and is miscible with water. During salt metathesis, evolution of CO₂ gas was monitored to assess the completion of the reaction. The resulting deep eutectic ionic liquid possessed both fluidity and transparency at room temperature with approximately 13% water content. The solution is a clear, colorless to yellow colored viscous liquid with a pH ˜8.5, conductivity ˜1.3 mS/cm, and a characteristic odor. Reaction duration and process parameters were optimized to improve manufacturing scale-up with the end product quality, defined by the water content, pH, conductivity and appearance. The initial scaled formulation (>3 kg) was designed to produce CAGE_{1:2} with the desired homogeneity for non-clinical studies. CGB400 gel was manufactured by mixing the CAGE_{1:2} solution with water, propylene glycol as co-solvent, d-limonene as a fragrance, and hydroxypropyl cellulose as a gelling agent. The inactive ingredients used in the CGB400 gel are inert in nature and were chosen to minimize dissociation of CAGE_{1:2} or have minimal effect on the CAGE_{1:2} structure.

Five kg batches of CGB400 gel were manufactured at an experienced contract manufacturing organization (CMO) in California for the human studies. Preservatives are often needed to protect against the growth of microbes in the formulation. However, since CGB400 gel itself possessed antimicrobial properties due to the inherent antimicrobial activity of CAGE_{1:2}, the formulation did not require additional preservation; keeping it paraben-free. All excipients used were USP/NF grade, except the fragrance agent which is a food-grade material.

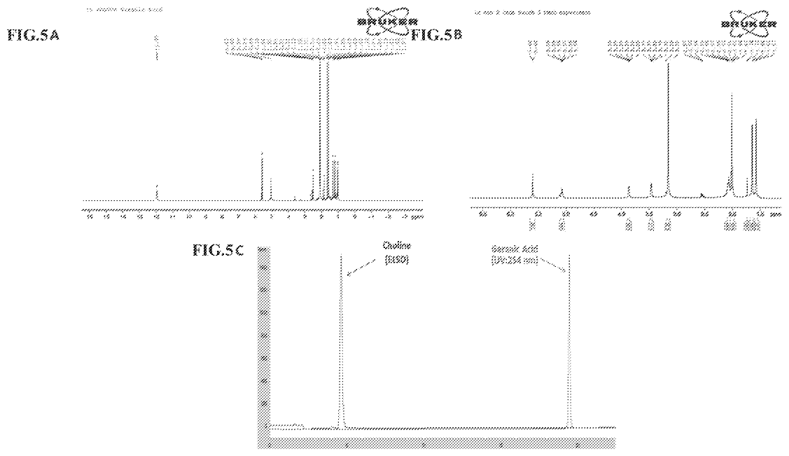

Of CAGE_{1:2}. Adequate characterization steps form the foundation of product development. For small molecules drugs, methods such as chromatography and NMR, among others, have been well-established over the years. As newer drugs such as biologics emerged on the landscape, the field of drug development has collectively rushed to develop the necessary methods for such products. This is one of the key challenges for deep eutectic solvents/ionic liquids. As an emerging category of actives, methods for characterizing these materials as drug products are in infancy. A key challenge is that the standard chemical methods are not adequate since no new covalent bond is formed during the salt metathesis between choline and geranic acid, thus making over reliance on conventional methods characterization challenging. CAGE_{1:2} was chemically characterized using NMR and HPLC analyses (FIGS. 5A-5C). ¹H NMR spectra of CAGE_{1:2} indicated notable differences in the chemical shifts compared to the individual components. These chemical shifts are pronounced for the protons localized adjacent to the quaternary nitrogen atoms indicating the strong anion effect of the anion, geranic acid. HPLC did not exhibit a clear signature of CAGE compared to its individual components. Specifically, the retention times (RT) of major peaks in the chromatogram of CAGE_{1:2} corresponded to that obtained in choline and geranic acid standard solutions. Both methods confirmed the presence of choline and geranic acid in CAGE_{1:2}.

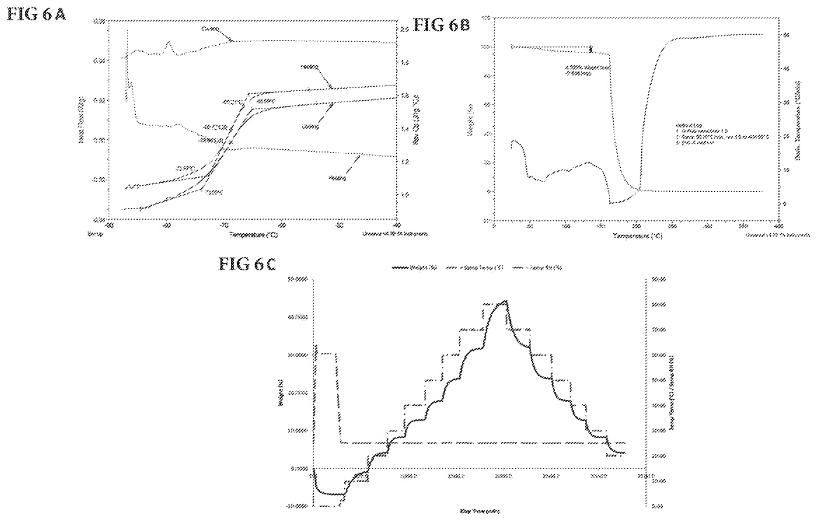

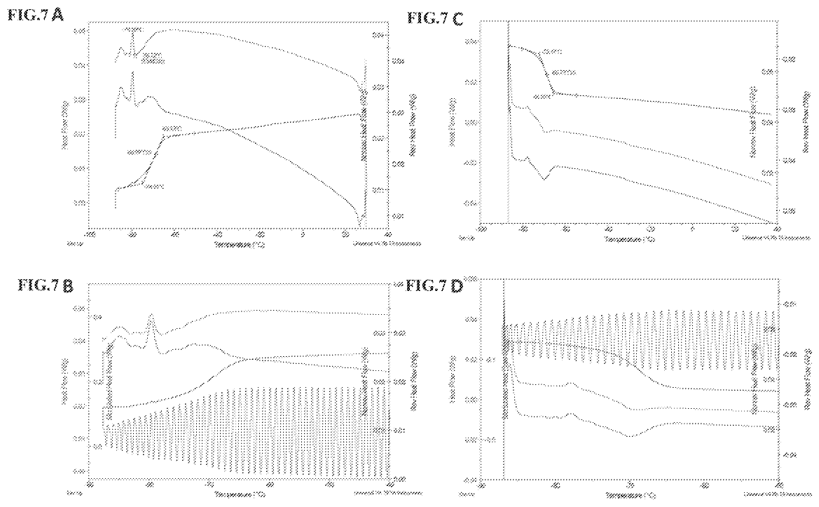

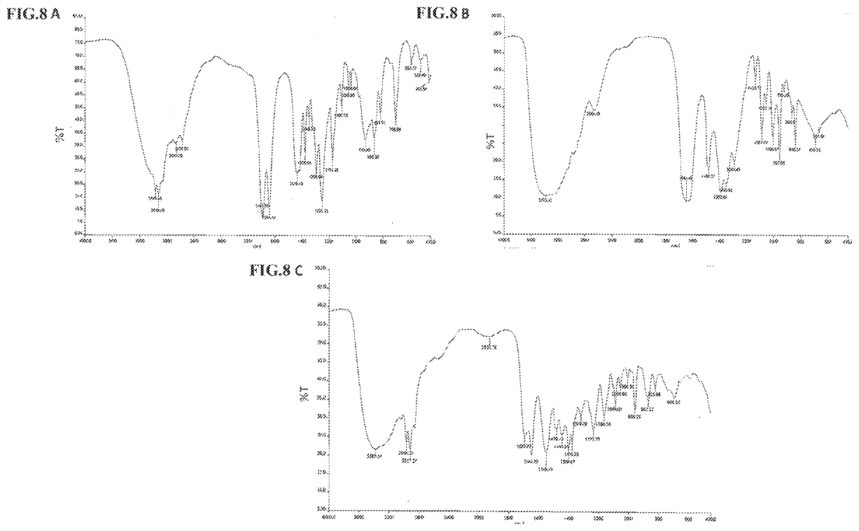

The physical and chemical properties of CAGE_{1:2} were additionally evaluated using modulated differential scanning calorimetry (MDSC) and thermal gravimetric analysis (TGA) and Fourier Transform Infrared Spectroscopy (FTIR). MDSC indicated a glass transition temperature (Tg) around −68° C. (FIGS. 6A-6C). This is illustrated in both the reversible heat flow and reversible heat capacity curves in both heating and cooling. It is likely that the liquid CAGE_{1:2} formulation solidifies into an amorphous solid. The small exotherm during cooldown at −80° C. (only apparent at 1° C./min) might be associated with an ordering event to an extent, like partial crystallization (FIGS. 7A-7D). From the TGA data, it is evident that CAGE_{1:2} lost approximately 4% weight at approx. 140° C., likely water. The sample completely evaporated or decomposed starting around 165° C. No hysteresis was observed in the sorption/desorption isotherm of the gravimetric vapor sorption which indicated that CAGE_{1:2} is hygroscopic (FIGS. 6A-6C). The salt metathesis reaction was also monitored via Fourier transform infrared (FT-IR) spectroscopy (FIGS. 8A-8C). The broad peak at 3300 cm⁻¹ most likely corresponds to the combination of choline —OH bond and carboxylic acid in geranic acid as is evident in CAGE_{1:2}. Additionally, the presence of peaks at 1690 cm⁻¹ and 2968 cm⁻¹ due to C═O and OH stretch of carboxylic acid in choline geranate confirms the presence of individual components of the ionic liquid.

In case of CAGE_{1:2}, it has been observed that partial dissociation of the anion and cation occurs in the presence of water. We anticipate that a dynamic equilibrium exists between CAGE_{1:2}, individual components and water. At concentrations below 20%, CAGE_{1:2} transitions from a bulk deep eutectic/ionic liquid structure into vesicles and transitions to microemulsion at much lower concentrations.

Stability under forced degradation conditions. Long-term stability of the drug product is a key requirement. It should be noted that the components of CAGE_{1:2} i.e. choline and geranic acid are GRAS materials with well-established safety profiles. No specific toxic impurities in either of these materials have been reported. Impurities in CAGE_{1:2} and CGB400 were quantified using validated HPLC methods. The limit for each impurity obtained at specific RT was monitored at lot release and during the stability studies. Stability of CGB400 was tested under forced degradation conditions following standard protocols. CGB400 formulation, upon exposure to heat or UV-A stress demonstrated minimal change in stability with a 0.6% and 0.4% loss of choline peaks respectively. A few new unknown peaks appeared in the chromatographic profiles of vehicle as well as the sample after heat and UV-A stress, but they did not interfere with the determination of choline. Similarly, for geranic acid the heat-, UV-, acid-, base- or peroxide-stressed vehicle contained no peaks that interfered with the determination of geranic acid. The heat-, UV-, acid-, base- or peroxide-stressed CGB400 gel showed a 20.0%, 2.0%, 0.3%, 0.0% and 3.6% loss of geranic acid respectively.

Solution stability of CGB400 gel was also tested. The stored samples and standards met the 95.0 to 105.0% recovery of initial concentration of choline and 97.0-103.0% recovery of initial concentration of geranic acid per the validation protocol criteria. The chemical and physical stability of CGB400 gel was evaluated over a 12-month period. The CGB400 gel was found to be stable in accelerated (40 C/75% RH), intermediate conditions (30 C/65% RH), and long-term storage conditions (25 C/60% RH) with relatively low increase in impurities level (total impurities level was increased from 0.42% to 2.5%). Moreover, microbial limit that was tested at every time point of the stability study was found to be in the acceptance range for topical gel products suggesting that CGB400 gel is well preserved for a period of at least 12 months. There was a slight increase in the pH (˜0.4) and change in viscosity (˜1000 cP) of the CGB400 gel at intermediate and RT conditions over a 12 months period. It is noteworthy that the change in viscosity observed over 12 months is not unusual for hydroxyethyl cellulose gels without a stabilizer. Inclusion of an antioxidant can potentially address this limitation. Additionally, no significant variation in the physical appearance, color and odor was observed. The minor changes in the visual appearance of the gel continued to meet specifications. We anticipate that the addition of an antioxidant would address the slight color change and viscosity drop of CGB400. The packaging for the GLP and cosmetic studies was performed in amber glass bottles.

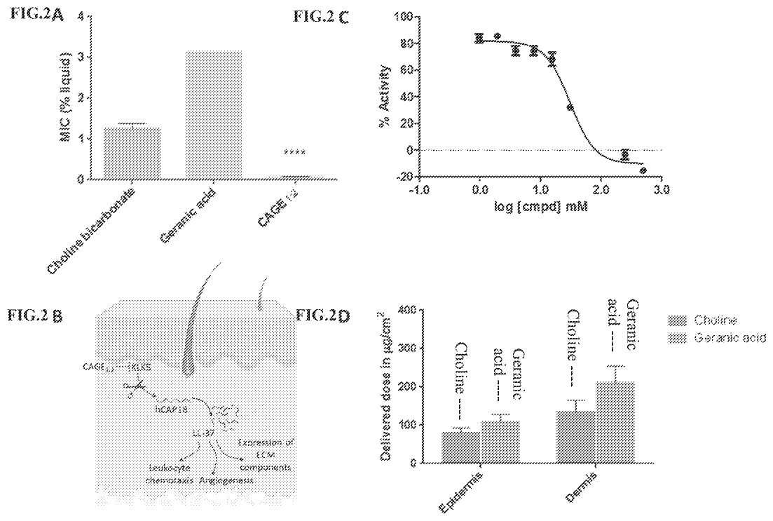

Mechanism of Action of CAGE_{1:2}: An understanding of the mechanism of action can enable making rational choices when multiple dosing or formulation options are feasible. The biological origin of rosacea is relatively less understood. However, studies have demonstrate the role of resident bacteria and the activity of kallikrein 5 enzyme (KLK5). Previous studies have demonstrated excellent antiparasitic efficacy of CAGE_{1:2} against a breadth of bacterial, fungal and viral species. The ability of CAGE_{1:2} to inhibit P. acnes as a model gram positive bacterium was tested using the CLSI standard broth microdilution methodology (BMD) (described in SI). CAGE_{1:2} demonstrated high antibacterial activity against P. acnes isolates (FIG. 2A). The Minimum Inhibitory Concentration (MIC) of CAGE_{1:2} ranged from 0.025-0.2% of neat CAGE_{1:2} (or approximately 57 μM-0.45 mM of CAGE_{1:2}). Without wishing to be bound by any particular theory, these results demonstrate the potential of CGB400 as an effective antimicrobial agent to treat underlying pathogenic origins in rosacea. In some embodiments, the MIC of CAGE_{1:2} was significantly lower than that of its individual components choline bicarbonate and geranic acid (P<0.0001), thus indicating that the effect of CAGE_{1:2} on microbial activity is not an additive effect of the individual antimicrobial activity of choline or geranic acid, but an effect that is attributable to the composition of CAGE_{1:2} under certain circumstance. In some embodiments, at least some portion of CAGE_{1:2} maintains the properties of a deep eutectic/ionic liquid and exhibits far superior antimicrobial activity compared to the individual components at the same concentrations.

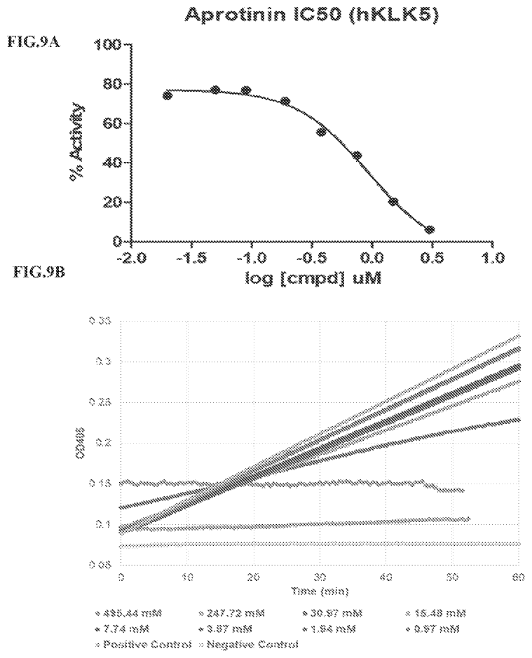

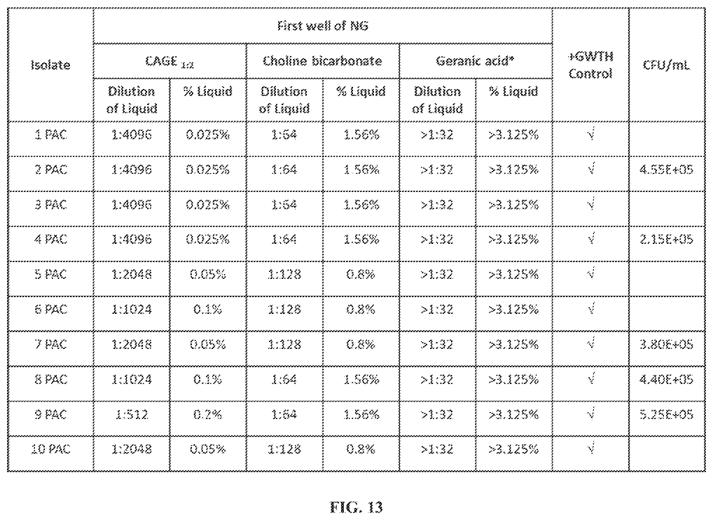

In some embodiments, CAGE_{1:2} also inhibited kallikrein 5 enzyme (KLK5). Effect of CAGE_{1:2} on hKLK5 was studied and compared to aprotinin as a positive control. IC₅₀ values of CAGE_{1:2} and Aprotinin were found to be 30.84 mM and 0.92 μM, respectively, indicating that CAGE_{1:2} is effective in inhibiting hKLK5 activity, although its potency is lower than that of aprotinin (FIGS. 2B and C, FIGS. 9A-9B, and FIG. 13).

Determination of dosing of CGB400. Mechanistic studies pointed out effective concentrations of <0.45 mM for antibacterial activity and 30.84 mM for KLK5 inhibition. In some cases, it is desirable to achieve the effective concentrations of KLK5 inhibition deeper into the skin including the epidermis and the dermis. Towards that goal, the permeation of choline and geranic acid from CGB400 gel into skin was evaluated in vitro using human cadaver skin. Since no covalent bond is formed between choline and geranic acid during the synthesis of CAGE_{1:2}, choline and geranic acid were separately detected in the skin. CGB400 gel exhibited excellent skin penetration generating high concentrations of its constituents, choline and geranic acid in the skin. The delivered doses of choline in the epidermis and dermis were 80.2±13.0 and 134.6±30.9 μg/cm² respectively, and the delivered doses of geranic acid were 109.3±19.5 and 210.7±44.3 μg/cm² in the epidermis and dermis respectively (FIG. 2D). The molar ratios of choline to geranic acid in the epidermis was ˜1.18 and that in the dermis was ˜1.03 at the end of 24 hours. A molar ratio of choline:geranic acid of 1.18 in the epidermis, though higher than that in the formulation (0.5), is indicative of the association of choline and geranic acid during the skin penetration of CAGE_{1:2}. Specifically, choline and geranic acid have substantially different lipophilicities (Log P of geranic acid, 2.8 and Log P of choline, −3.7). Accordingly, their individual skin permeabilities are expected to be dramatically different. Hence, the closeness of the choline:geranic acid molar ratio in the skin to that in the applied formulation suggests the existence of interactions between choline and geranic acid during skin penetration. Penetration of CAGE into skin had a quick onset. Specifically, the fluxes of choline and geranic acid in the epidermis after 8 hours of formulation placement were 3.48±0.55 and 27.54±1.94 μg/cm²/h respectively. An epidermal choline dose of 80.2±13.0 μg/cm² leads to an estimated epidermal CAGE concentration of ˜76 mM assuming an epidermal thickness of 100 μm, a number in the target range of therapeutic activity.

GLP dermal toxicity in minipigs. Minipigs are considered an valid animal model for studying toxicity of topical skin formulations. GLP toxicity studies were performed in Göttingen minipigs by applying the CGB400 gel once a day for 91 consecutive days. 1 ml CGB400 was applied over 10% of the total body surface area of the animal. All animals survived to scheduled study termination and there were no macroscopic findings. No clinical signs, changes in body weight, food consumption, ocular, electrocardiography, hematology, coagulation, clinical chemistry, urine analysis, organ weights parameters were observed. Dermal changes including edema, erythema, fissuring and focal red spots were observed in CGB400-treated animals and completely disappeared by the end of 14-day recovery period. Overall, CGB400 was well tolerated in minipigs.

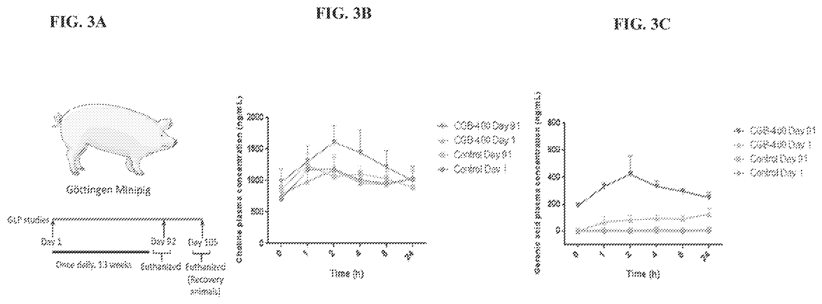

Toxicokinetic parameters were determined from plasma concentration of choline and geranic acid. The mean±std.error Cₘₐₓ values for choline on Day 1 for male and female were 609±169, 459±106; and on Day 91 were 783±196, 1080±492 ng/ml respectively. Besides, the AUC0-Tlast on Day 1 for male and female were 4610±3570, 5350±1680; and on Day 91 were 6750±2650, 3110±776 hr*ng/mL respectively. Over the 91-day treatment period, Cₘₐₓ and AUC accumulation ratios of choline (Day 91/Day 1) were 1.3 and 1.5 respectively in males and 2.4 and 0.6 in females, demonstrating accumulation to a certain extent when CGB400 gel was applied by dermal administration to Göttingen minipigs daily for 13 consecutive weeks. It is worth noting that the choline levels measured are not exclusive to the exogenous choline given to the animals through the CGB400 gel administration, as they were levels of choline ingested through the feeding as well as endogenous levels of choline. In fact, the choline levels for the control group (treated with a vehicle containing ˜90% water along with propylene glycol, a fragrance, and a gelling agent) are indicative of the baseline levels of choline in plasma. In contrast, the mean Cₘₐₓ values for geranic acid on Day 1 for male and female were 197±64.1, 68.5±24.3; and on Day 91 were 373±44.2, 573±254 ng/ml respectively. In addition, the AUC0-Tlast on Day 1 for male and female were 3560±938, 1260±459; and on Day 91 were 7050±826, 6950±1390 hr*ng/mL respectively. Cₘₐₓ and AUC accumulation ratios of geranic acid (Day 91/Day 1) were 1.9 and 2.0 respectively in males and 8.4 and 5.5 in females, suggesting consequential accumulation of geranic acid. The plasma concentrations of choline and geranic acid on Days 1 and 91 in comparison to control following dermal administration have been depicted in FIG. 3.

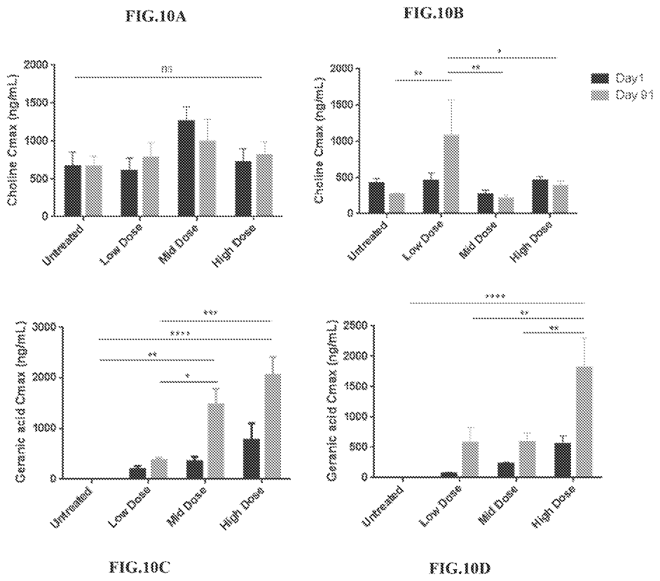

Toxicokinetic studies in pigs also included two additional doses, one at 1.5× of CGB400 (that is, 60% CAGE_{1:2}) and one at about twice that of CGB400 (that is, 87% CAGE_{1:2}, which is pure CAGE_{1:2} which naturally absorbs water to form a 87% mixture). Generally similar results were obtained at these doses and the plasma concentrations of geranic acid generally scaled with the dose (FIGS. 10A-10D).

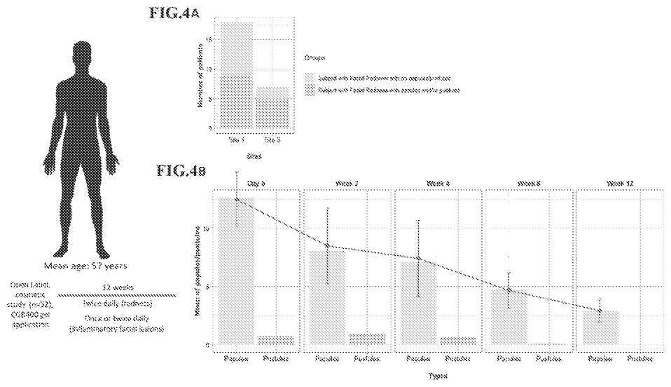

Clinical evaluation in human volunteers. In an IRB-approved 12-week open label cosmetic study for rosacea, subjects with a mean baseline lesion count (papules and pustules) of 13.4±6.7 lesions were presented. Three sites were used for the study and the median age of subjects was 57 years (FIG. 4A). After 2 weeks of therapy with CGB400 gel, the mean papules and pustules count was reduced by 34.6% (p=0.0018) with further reductions in lesion counts being observed at each subsequent study visit. The greatest reduction in lesion counts was seen at week 12 (visit 5) where a mean drop from baseline of 71.9% (P<0.0001) was observed (FIG. 4B).

It is worth noting that the CGB-400 gel dose applied daily on the minipig's skin was 1 g, where each gram of gel contained 0.4 g of CAGE. At the highest concentration (87%), the applied dose of CAGE_{1:2} was 0.87 g per day, which is equivalent to 69.3 mg/kg/day, assuming 12.55 kg as an average weight of a minipig. For the studies in human volunteers, ˜0.2 g of a drug product was applied twice a day i.e. 0.08 g of CAGE_{1:2} per application or 2.3 mg/kg/day for a 70 kg adult. In some embodiments, the target human dose is ˜30× lower than the highest dose evaluated in the repeat dose toxicity study in minipigs.

In addition, investigator global assessment (IGA) responder rates (i.e., ≥1-point decrease from baseline) as well as the proportion of subjects with IGA scores of “clear” or “almost clear” were assessed. Both IGA measures demonstrated accumulated effects throughout the study. Therapeutic response was noted as early as visit 2 (week 2), where 44.4% [25.5, 64.7] of subjects were deemed to be IGA “responders”, and 29.6% [13.8, 50.2] of subjects were clear/almost clear. Response continued to strengthen out to visit 5 (week 12) where 82.6% [61.2, 95.1] of subject were deemed to be IGA “responders”, and 69.6% [47.1, 86.8] of subjects were clear/almost clear.

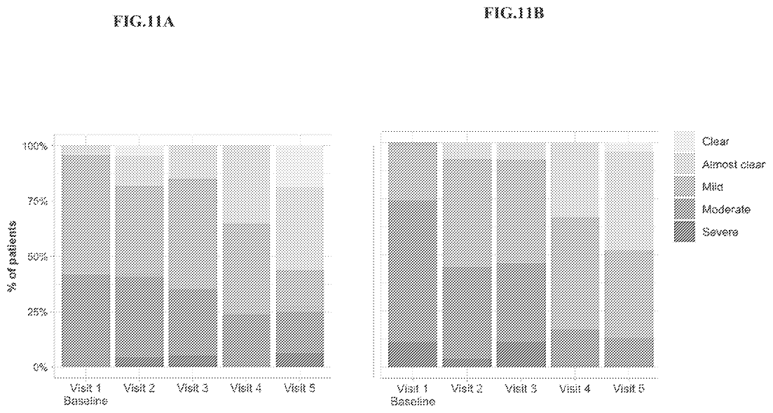

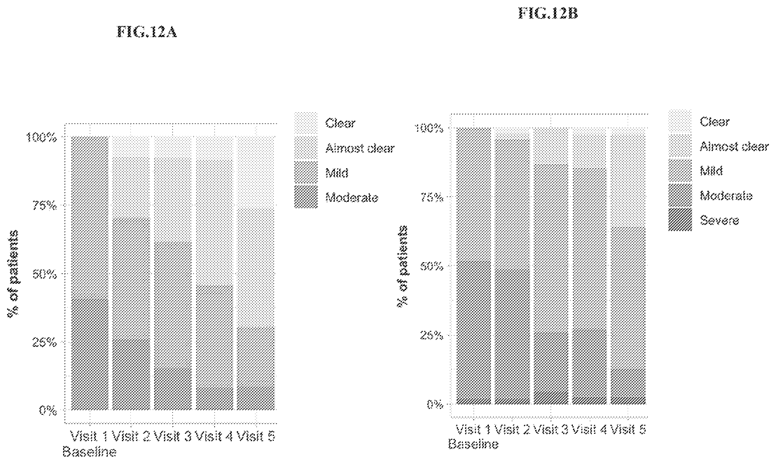

Furthermore, IGA-Redness (IGAR) responder rates (i.e., ≥1-point decrease from baseline) as well as the proportion of subjects with IGAR scores of “clear” or “almost clear” were assessed. Both IGAR measures demonstrated accumulated effects throughout the study. IGA captured investigators assessment of inflammatory lesions on the face whereas IGAR specifically captured facial redness, independent of lesions. Therapeutic response was noted as early as visit 2 (week 2), where 22.2% [8.6, 42.3] of subjects were deemed to be IGAR “responders”. However, only 3.7% [0.1, 19.0] of subjects were clear/almost clear. Response continued to strengthen out to visit 5 (week 12) where 69.6% [47.1, 86.8] of subject were deemed to be IGAR “responders”, and 47.8% [26.8, 69.4] of subjects were clear/almost clear. Likewise, in patients without bumps/blemishes, 50.0% [27.2, 72.8] and 52.9% [27.8, 77.0] of subjects were deemed to be IGAR “responders” at visit 2 (week 2) and visit 3 (week 4), respectively. Effectiveness evaluations based on PGA, IGA and IGAR in subjects with facial redness with/without bumps/blemishes (FIGS. 11A-11B and FIGS. 12A-12B).

Patient Global Assessment (PGA) responder rates (i.e., ≥1-point decrease from baseline) were also assessed. After only 2 weeks of treatment, the majority of subjects (i.e., 51.9% [32.0, 71.3]) were already PGA responders. Response continued to strengthen out to visit 5 (week 12) where 73.9% [51.6, 89.8] of subjects were deemed to be PGA “responders”. Likewise, for patients without bumps/blemishes, after 2 weeks of treatment, 6 (31.6% [12.6, 56.6]) subjects were PGA responders and the response strengthened out to visit 3 (week 4) where 50.0% [24.7, 75.4] of subjects were deemed to be PGA “responders.”

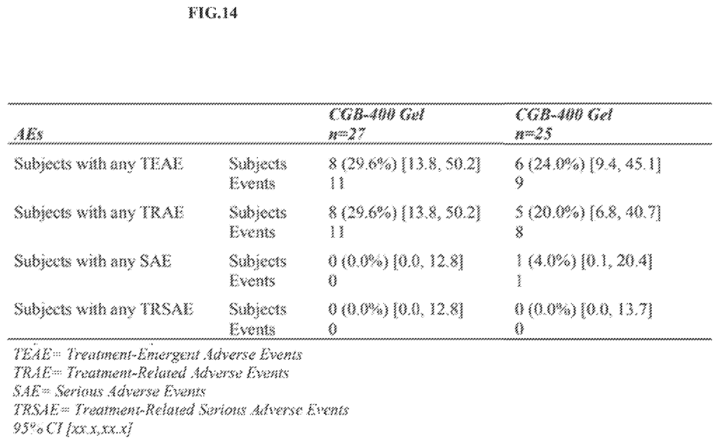

Safety assessment indicated 8 (29.6% [13.8, 50.2]) subjects with bumps/blemishes experienced a total of 11 Treatment-Related Adverse Events (TEAE). There were no SAEs reported in the study. Moreover, 6 (24.0% [9.4, 45.1]) subjects experienced a total of 9 Treatment-Emergent Adverse Events (TEAE), and 5 (20.0% [6.8, 40.7]) subjects experienced a total of 8 Treatment-Related Adverse Events (TRAE). One SAE (unrelated to study treatment) was reported by 1 subject (Heart Valve Replacement) in patients without bumps/blemishes (FIG. 14).

### Example 2. Materials and Methods

One-step lab scale formulation of CAGE_{1:2}. Choline geranate (CAGE_{1:2}) was synthesized as described previously. Briefly, the cation, choline bicarbonate and geranic acid, anion was mixed at a 1:2 ratio to prepare IL/deep eutectic mixture following salt metathesis reaction. Geranic acid (3.696 mol) was weighed in a stainless-steel vessel equipped with a stirrer and placed into a water bath. Choline bicarbonate 80% solution (1.848 mol) was added dropwise to the vessel. The mixture was reacted for 8 h at 27° C. The water content of the resulting IL/deep eutectic mixture was measured using Karl Fischer (KF) titration as an in-process parameter.

CAGE_{1:2} characterization. Thermal analysis (mDSC and TGA) and moisture sorption analysis (DVS) of CAGE_{1:2}, eutectic mixture was carried out by CeutixLabs, San Diego. The method development for modulated differential scanning calorimeter (mDSC) and TGA have been discussed in detail (Supplementary materials and methods). For the mDSC, sample was dried for 3 hours at 60 C on the TGA prior to running the DSC. The sample was quickly sealed (hermetically) after drying on the TGA which helped in removing most of the moisture and water associated thermal events (there may be a very low fraction of water left, i.e. <0.5%). There were two parts to the MDSC analysis including run at 3 C/min followed by run at 1 C/min. The run at 1 C/min was better controlled over a wider range. It should be noted that the modulation signal gets a little distorted <−80 C during the cooling cycle, so doesn't read the fine details below −80 C. The TGA was ran in Hi-Res mode. i.e. a dynamic (variable) heating rate where the heating rate is inversely proportional to the weight loss rate. On the Y-2 axis of the TGA plot, the derivative of the temperature vs time was plotted. This was simply to illustrate the instantaneous heating rate (degrees/min) at any particular temperature during the run. This shows that the dynamic heating rate varied from nearly 0 (around 160 C—when the weight dropped off the cliff) to 50 C/min near the end of the run. Dynamic Vapor Sorption (DVS), using gravimetric moisture sorption (GVS) analysis was also conducted. Both TGA and GVS analyses have been considered since, TGA measures volatiles in % wet weight, whereas GVS measures water uptake with respect to dry weight. Additionally, FTIR analysis of choline, geranic acid and CAGE_{1:2} were carried out and compared using FTIR Spectrometer, Perkin-Elmer Spectrum One. Report builder, Rev 2.01 (software) was used for spectra analyses.

Anti-microbiological in vitro activity. CAGE_{1:2} and its individual components were tested for any antibacterial activity against selected organisms, using the CLSI standard broth microdilution methodology (BMD). Clinical isolates of Propionibacterium acnes (newly classified as Cutibacterium acnes) were employed in this study. Considerable precipitate was observed when each of the test components was added to the media at a 1:2 dilution. Therefore, preliminary concentration checks were performed to determine the highest concentrations of the liquid to be utilized.

hKLK5 Enzymatic Assay. Test compound CAGE_{1:2} (87.07% w/v) was diluted to 0.99 mM and control compound Aprotinin was prepared at 6 μM in di-water. A 2-fold serial dilution was then performed in di-water to generate 10 concentrations for CAGE_{1:2} (87.07% w/v) and 8 concentrations for Aprotinin which were 2× of the final concentrations in the reaction. Recombinant human KLK5 was purchased from Speed Biosystems LLC (Catalog No. YS1485, Lot No. 0330517, 17.2 μM). The substrate S-2288 was purchased from Diapharma Group, Inc (Catalog No. S820852, Lot No. N0889942).

The assay was carried out in 384-well clear bottom plate coated with 0.1% Tween 20 at 4° C. overnight and washed twice with di-water before usage. 50 μL 2× compound in water was mixed with 25 μL 4× hKLK5 enzyme (120 nM) prepared in 2× reaction buffer (100 mM Tris, pH 8.0, 200 mM NaCl, 0.2% PEG, 0.01% Tween 20, 2 mM EDTA). The enzymatic reaction was initiated by the addition of 25 μL 4× substrate S-2288 (2 mM) in 2× reaction buffer to all wells. OD 405 nM was measured every 30 seconds by SpetraMax 384 Plus at 37° C. for 1 hour.

Human cadaver skin permeation evaluation. Dermatomed full thickness human cadaver skin samples, 250-350 μm, were mounted onto static Franz cells. The cells had a contact area of 0.7 cm² and a receptor volume of 4 ml. Phosphate buffered saline (PBS), pH 7.4 was used as the receptor medium. CGB400 gel was applied to the stratum corneum side of the skin samples at a dose of ˜15 μL/cm² (˜15 mg/cm²). The cells were kept in a chamber at 37 C for a period of 24 h. Stirrers in the receptor chamber ensured that the receptor media was well mixed during this time. The receptor media was sampled at 4, 7, and 24 hours to quantify the amount of choline and geranic acid permeation. After 24 hours, the skin surface was washed before removing the stratum corneum (SC) by tape stripping. The SC was stripped from the epidermis using an adhesive tape up to ten layers (SC1, SC2-5, SC6-10). Following SC removal, the epidermis was separated from the dermis using a surgical sterile scalpel and the dermis was cut into pieces. Each layer was collected separately in glass vials containing 2 mL of 50:50 water:methanol mixture and was left to shake overnight to extract the choline and geranic acid. The amount of choline and geranic acid was measured using LC-MS/MS in the wash, epidermis, dermis, and the receptor medium.

Forced degradation and stability validation studies. The content of choline and geranic acid in CGB400 gel following stability and forced degradation studies were analyzed via matrix-matched standard method using Agilent technologies 1100 and 1200 Series HPLC. System suitability, standard and method precision, linearity, accuracy/recovery and specificity were successfully established. Specificity by forced degradation was performed by stressing samples of the vehicle and CGB400 topical gel formulations. Samples were stressed with heat at 80° C. oven and UV-A light for 5 days. The heat and UV-A stressed vehicles contained no peaks that interfered with the determination of choline. Specificity was demonstrated by comparison of the chromatographic profiles of active and stressed vehicle samples to demonstrate that no vehicle-related degradation products elute in the retention window of choline. The loss of analyte was evaluated by the decrease in relative peak area of choline after the stress exposure. The purity factor was then calculated for the heat- and UV-A-stressed CGB400 gel. An overall purity factor of 999.85 and 999.87 for the choline peak was observed in this sample respectively, indicating the absence of coeluting compounds with dissimilar spectra from choline.

Similarly, for geranic acid, samples were stressed with heat at 80° C. oven for 5 days, UVA light for 3 days, 0.1 M hydrochloric acid for 24 hours, 0.1 M sodium hydroxide base for 24 hours, and 3% hydrogen peroxide for 24 hours. No peaks appeared in the chromatographic profiles of UV-, acid-, base- and peroxide-stressed samples other than heat-stressed samples, where few new peaks appeared. Specificity was demonstrated by comparison of the chromatographic profiles of active and stressed vehicle samples to demonstrate that no vehicle related degradation products elute in the retention window of geranic acid isomers. In addition, the geranic acid peaks in the stressed active samples were evaluated by peak purity. Peak purity was evaluated from 200 to 300 nm for choline and 239 to 279 nm for geranic acid, bracketing each side of the 208 nm and 254 nm detection wavelength respectively. Spectra throughout the peak were evaluated at a similarity threshold of 999 to assure that no impurity(s) with dissimilar spectra greater than 0.1% of the analytical concentration were present in the geranic acid peaks. A numerical value for this uniformity was calculated as the peak purity factor. A value of 1000 indicates no variation in the spectra for the peak. The purity factors were calculated for each geranic acid isomer in the heat, UV, acid, base and peroxide-stressed 40% CG-101 gel sample.

Evaluation of the spectral data collected from the photodiode array detector was performed to determine the purity of the choline and geranic acid isomer peaks. The spectra from 200 to 300 nm for choline and 239 to 279 nm for geranic acid, at five time points (peak maximum, two points before the maximum and two points on the peak tail) were compared to each other.

For solution stability, standards and samples were prepared and analyzed on the day of preparation. For choline, the solutions were stored in sealed vials at room temperature, then re-analyzed at 72 hours against freshly prepared standards. For geranic acid, the solutions were stored in sealed flasks at 5° C. and room temperature, then re-analyzed after 48 hours against freshly prepared standards. The validation data showed that standards and samples had stable choline levels after being stored at room temperature at least 72 hours after preparation. Similarly, standards and samples for geranic acid were stable for at least 2 days after preparation when stored under refrigerated conditions.

Determination of dermal toxicity and toxicokinetic (TK) profile in minipigs. For GLP studies, the test and control/vehicle items were administered to groups of minipigs daily by non-occluded dermal application for 13 weeks or 91 consecutive days. The volume of test or control/vehicle items (1 mL) was applied to the dermal test site and spread uniformly over the surface of the skin, using a gloved finger but ensuring that excessive residue did not remain on the glove. Gloves were changed between each group of animals. The same dermal test site was used for all doses. Assessment of toxicity was based on mortality, clinical observations, body weight, food consumption, dermal changes, ophthalmology, electrocardiography, and clinical and anatomic pathology.

During the GLP dermal toxicity study in gottingen minipigs, a series of 6 blood samples (approximately 0.5 mL each) was collected by venipuncture from each animal on Days 1 and 91 of the treatment period at the following timepoints: Pre-dose, 1, 2, 4, 8, and 24 hours after treatment. The samples were collected into tubes containing K₂EDTA as an anticoagulant. Tubes were placed on wet ice pending processing. Following collection, the samples were centrifuged (1000 g for 10 minutes at approximately 4° C.) and the resulting plasma was recovered, divided into two aliquots (sets 1 and 2) of approximate equal volume in appropriately labelled tubes and placed on dry ice pending storage in a freezer (≤−60° C.). Blood samples were analyzed at a GLP bioanalytical lab in CA.

Following dosing, main animals were euthanized and subjected to a necropsy examination on Day 92. Recovery animals were observed for 14 days and then euthanized and subjected to a necropsy examination on Day 105. Animals from each group were assigned to different replicates for logistical reasons.

Phase Ib cosmetic study design. The goal of this study was to evaluate the ability of CGB400 in reducing facial redness, bumps/blemishes (inflammatory lesions such as papules and pustules) and determine its safety and tolerability. A multicenter, open-label, single-group proof-of-concept study using CGB400 Gel to reduce facial redness, bumps, and blemishes was conducted. Twenty-five subjects with redness, but without bumps/blemishes, received study treatment for 4 weeks and attended a total of 5 study visits (i.e., BL, W1, W2, W4, and post-treatment follow-up at W5). Twenty-seven subjects with redness and bumps/blemishes received study treatment for 12 weeks and attended a total of 6 study visits (i.e., BL, W2, W4, W8, W12, and post-treatment follow-up at W13. CGB400 gel was applied topically to the face twice per day, once in the morning after a shower or washing the face, and once in the evening after washing the face. It was a proof-of-concept (POC) study and a formal sample size justification was not provided. It was the opinion of the sponsor that 40-50 subjects would be sufficient to achieve study objectives.

Diagnosis for inclusion included:

Subjects with facial redness and bumps/blemishes:
Facial redness associated with rosacea.
Facial redness (IGAR) score of 2 or 3 (i.e., mild or moderate).
IGA score of 2 or 3 (i.e., mild or moderate).

Subjects with facial redness and without bumps/blemishes:
Facial redness associated with rosacea.
Facial redness (IGAR) score of 2 or 3 (i.e., mild or moderate).

Effectiveness evaluations were based on:
Investigator's Global Assessment (IGA)
Investigator's Global Assessment of Redness (IGAR)
Bumps/Blemishes Count
Subject or Patient Global Assessment (PGA)

Safety evaluations were based on:
Adverse events

Statistical Methods:

Analyses for effectiveness and safety were conducted on a modified intent-to-treat (mITT) basis. The mITT population consisted of all subjects who received at least one application of CGB400 gel and provided at least one post-baseline evaluation.

All statistical processing was performed using SAS® version 9.4. For categorical parameters, the number and percentage of subjects/observations in each category were presented. The denominator was based on the number of subjects/observations appropriate for the purpose of analysis. For continuous parameters, descriptive statistics included n (number of subjects or observations), mean, standard deviation, median, and range. 95% Confidence Intervals (CI) were provided for all efficacy outcomes.

### Example 3: Materials and Methods

Differential Scanning calorimeter (DSC). Empty DSC pan and lid (hermetic Tzero) on the analytical balance were weighed. Weights were recorded and tared. Using a spatula, approximately 25 mg (viscous liquid) was transferred into the pan. The open DSC pan was then transferred to the TGA and dried for 3 hours at 60° C. (N2 flow was set at 100 mL/min for the sample). After drying, the sample was hermetically sealed using the crimper and hermetic die set. The sealed sample pan was weighed again on the analytical balance and the fill weight was calculated by subtracting the DSC pan and lid weights. The pan weights and fill weights were entered into the sample parameter section of the Q-Series control program for the Q2000 DSC. The N2 purge flow rate was set to 50 mL/min. One cool/heat run between −85° C. and 20° C. was performed using a heating ramp of 3° C./min. Modulation was set at +/−1.00° C. every 60 seconds. A second run was performed between ca. −88° C. and 30° C. using a heating ramp of 1.00° C./min. Modulation was set at +/−1.00° C. every 60 seconds.

Thermogravimetric analysis (TGA). The sample was prepared by weighing approximately 16 mg of material into a TA Pt pan. Samples were heated in the Hi-Res mode as per the method below. Nitrogen purge was set at 40 ml/min for the balance and 60 ml/min for the furnace. Data were analyzed using the TA Universal Analysis 2000 software (version 4.7B).

Broth microdilution assay. Serial dilutions were made in Brucella broth+5% lysed horse blood at concentrations ranging from 6.25% liquid (1:8 dilution)−0.0003906% liquid (1:262,144 dilution). The liquid/broth dilutions were dispensed (100 μL/well) into 96 well microtiter trays. A positive control well was filled with 100 μL of broth only. Bacterial suspension was made in sterile pre-reduced Brucella broth, to a density equivalent to that of a 0.5 McFarland standard (108 CFU/mL). Inocula were prepared from blood agar plates incubated for 48 hours. Each bacterial suspension was transferred to 10 mLs of suitable media as follows: 500 μL of P. acnes (C. acnes) was transferred to Brucella+5% LHB. Each of the final bacterial suspensions was poured into a sterile trough and 10 μL was dispensed into each well of the microtiter tray. Colony counts were determined from the positive growth well by removing 20 μL and diluting in 10 mLs of sterile normal saline. The tube was inverted several times to mix and a 100 μL aliquot was used to inoculate a blood agar plate (BAP). The colony count plates, and MIC trays were incubated under specified conditions and growth observed after 48 hours. MICs were read as the lowest concentration (% liquid) that inhibited growth of the test organism (based on turbidity). For Geranic acid, the highest 3 concentrations were subcultured to solid media to validate the turbidity. Colonies were counted on each colony count plate (100 colonies were equivalent to 5×105 CFU/mL, the desired bacterial concentration).

Suitability of AET by Plate Count Method. The dilutions of each challenge microorganism suspensions were prepared such that the resulting level was approximately 105 CFU per mL. The 1:10, 1:100 and 1:1000 dilution of the CGB400 placebo gel were prepared in TSB-M. A 1:10 dilution of the CGB400 placebo gel was prepared by adding 10 g of the CGB400 placebo gel to 90 mL of TSB-M. This dilution scheme was continued to achieve 1:100 and 1:1000 dilution levels.

An appropriate number of challenge organisms was added to each tube of the diluted test article and mixed well. Two empty petri dishes were labelled for each challenge organism and 1 mL of the inoculated CGB400 placebo gel dilution was plated into each dish to yield not more than 250 CFU/plate for P. aeruginosa, S. aureus, E. coli and C. albicans or not more than 80 CFU for A. brasiliensis. The volume of the suspension of the inoculum did not exceed 1% of the volume of diluted CGB400 placebo gel. In parallel, two control plates containing diluent without the CGB400 placebo gel were prepared for each organism. These served as positive controls. To show that the neutralizer is effective in inhibiting the antimicrobial properties of the CGB400 placebo gel (neutralizer efficacy) without impairing the recovery of viable microorganisms (neutralizer toxicity) a second set of positive controls using saline was prepared for each organism by inoculating the same volume of suspension dilution. Immediately after inoculation, 15-20 mL of agar appropriate for each challenge organism (TSA for bacteria, SDA for yeast and mold) was added to each dish. The plates were allowed to solidify before placing into the incubators. The TSA plates were incubated for not more than three days at 30-35° C. and SDA plates were incubated for not more than three days at 20-25° C. At the conclusion of the incubation period, the number of colonies present on each plate was counted and the average CFU for the two plates was calculated.

Method development and validation for choline in CGB400 gel. Calibration standards were consecutively injected six times at the beginning of each analysis. Standard precision was evaluated by calculating the relative standard deviation (RSD) of the choline peak area for the first six standard injections. The precision for choline peak areas in the first six injections ranged from 0.0% to 0.2%. The acceptance criterion of not more than 5.0% RSD, specified in the USP Choline Chloride monograph, will be kept. A duplicate check standard was prepared along with the calibration standard on each day of analysis. An injection of the check standard was performed, and the response factor of the check standard injection was compared to the mean response factor of the first six injections of the calibration standard. The choline response factor of the check standard agreed with the calibration standard response factor within the 95.0 to 105.0% acceptance criterion of the method. A blank preparation was injected as part of system suitability. The chromatograms for the blank were evaluated and no peaks larger than 0.5% of the mean area of first six injections of the calibration standard were observed in the retention window of choline. Peak shape was assessed for the choline peak in the last of the six injections of calibration standard. Peak shape attributes collected were peak tailing and theoretical plates. Based on the peak tailing results, an acceptance criterion of <2.0 will be set. Based on theoretical plates results, an acceptance criterion of ≥5,000 will be set. The choline signal-to-noise ratio was assessed for the sensitivity solution. For all analyses the S/N was ≥10.

Method development and validation for geranic acid in CGB400 gel. Calibration standard was consecutively injected five times at the beginning of analysis for system precision check. Standard precision was evaluated by calculating the relative standard deviation (RSD) of the geranic acid peak areas for the first five standard injections. The precision for geranic acid peak areas in the first five injections met the acceptance criterion of not more than 2.0% RSD on each day of analysis. A three-point calibration curve is obtained by plotting the total geranic acid peak area of single injections for 0.75, 0.5 and 0.25 mg/mL calibration standard solutions on each day of analysis. The geranic acid calibration curve correlation coefficient was 1.000 on each day of analysis, meeting the acceptance criterion of not less than 0.997. A blank preparation was injected as a part of system suitability. The chromatograms for the blank were evaluated and no peaks larger than 0.5% of the mean area of first 5 injections of the calibration standard were observed in the retention window of geranic acid.

Peak shape was assessed for the Geranic Acid 2 peak in the last injection of the 0.5 mg/mL calibration standard for system precision check. Peak shape attributes collected were peak tailing, theoretical plates, as well as the resolution between Geranic Acids 1 and 2. In all cases, the peak tailing was 1.1 and the theoretical plates greater than 250,000 were obtained for Geranic Acid 2 isomer. Resolution between the Geranic Acid 1 peak and the Geranic Acid 2 peak ranged from 1.51 to 2.09. Since the loss of peak efficiency and peak symmetry will directly result in loss of resolution, monitoring resolution will be a sufficient suitability test of peak shape. An acceptance criterion of no less than (NLT) 1.5 will be set for resolution. The signal-to-noise (S/N) ratio was assessed for the sensitivity solution. The S/N for all analyses was greater than 10.

To demonstrate the consistency of the instrument throughout the run, bracketing standards were injected after a maximum of six sample injections. The response factor of each bracketing standard injection was compared to the mean response factor of the first five injections of the 0.5 mg/mL calibration standard. The geranic acid response factor of each bracketing standard ranged from 97.0% to 100.1% with the calibration standard response factor. Therefore, an acceptance criterion of 97.0% to 103.0% will be proposed for the method.

Test system preparation and dosing in GLP minipig study. During the pre-treatment period (between Days −5 and −2), an area on the upper dorsum (across the midline) of each minipig was clipped free of hair. This area was limited in size from approximately the rump to the lower neck region and halfway down the flanks. The dermal test site was cleared of hair as often as necessary and care was taken to not damage the skin. Any damage caused mechanically was noted in the raw data. The dermal test site was defined by dots

Head

| | | | |
|---|---|---|---|
| Left | • | • | Right |
| | • | • | |

Tail

This figure is not drawn to scale

in each of the four corners using an indelible ink following the Day −1 body weight (to allow calculation of 10% Total Body Surface Area [BSA]). The area does not necessarily need to be a square; a rectangle is acceptable as long as the % BSA is adhered to.

The dermal test site (area of application) was calculated as 10% of the Total Body Surface Area of the animal (i.e. area of skin by the body weight) on Day −1 and weekly thereafter, using the following formula, which is specific to the minipig:

BSA (m2)=(BW (kg)*1000)\hat{ }(2/3)*k/10000
Where BSA=body surface area of the animal, BW=body weight and the value of k=9. (Formula by Meeh). If the dermal test site size increased during the study, the anchoring point was the left corner nearest to the head.

Washing of the dermal site in minipigs. On all dosing occasions, with the exception of Day 1, the dermal test site was wiped clean using water moistened gauze prior to dosing to remove any residue of formulation from the skin. Washing was performed using warm reverse osmosis water. Unscented soap was used if deemed necessary. The dermal test site was dried with soft tissue, using gentle pressure.

Toxicokinetic studies. A series of 6 blood samples (approximately 0.5 mL each) was collected by venipuncture from each animal on Days 1 and 91 of the treatment periods at the following timepoints: Pre-dose, 1, 2, 4, 8, and 24 hours after treatment. The samples were collected into tubes containing an appropriate anticoagulant (to be added via amendment). Tubes were placed on wet ice pending processing. Following collection, the samples were centrifuged (1000 g (approximately 2500 rpm) for 10 minutes at approximately 4° C.) and the resulting plasma was recovered, divided into two aliquots (sets 1 and 2) of approximate equal volume in appropriately labelled tubes and placed on dry ice pending storage in a freezer (≤−60° C.).

Bioanalytical method for determining choline and geranic acid in minipig plasma samples. Concentrations of Geranic Acid and Choline were determined in 373 Minipig plasma samples by an LC/MS/MS method. In the method, 10 μL of standard spiking solutions of Geranic acid of appropriate concentrations were added to individual 190 μL aliquots of blank minipig plasma for preparing Geranic Acid calibration standards and QCs. 120 μL of MeOH was added to 40 μL of plasma and centrifuged at 4000 rpm for 10 minutes. For choline, 64 μL of internal standard solution (choline-d9) was added to 16 μL H₂O. All the calibration standards and QCs were freshly made from stock solution stored at −20° C. freezer. The Shimadzu Prominence HPLC system (PMO-0198, 0163, 0156 and 0201) combined with AB Sciex API 5000 (PMO-0193) was used to perform the LC/MS/MS analysis of Choline, and the Shimadzu Prominence HPLC system (PMO-0197, 0162, 0188 and 0202) combined with AB Sciex API 5000 (PMO-0226) was used to perform the LC/MS/MS analysis of Geranic Acid. With a 2 μL and 10 μL of sample injection, Choline and Geranic Acid, respectively, were separated from interfering substances and subsequently eluted from the HPLC column for mass quantification. An MRM mode of mass spectrometer analysis was used to detect and quantify Geranic Acid and Choline. Data were processed and final concentrations of samples were calculated by an automated data acquisition and process system—Analyst 1.4.2 (Applied Biosystems, Foster City, CA).

### Example 4: CAGE 1:2 Formulation

TABLE 1

| | | | | Quantity for | |
|---|---|---|---|---|---|
| | | | Quantity for | CGB-400 Low | Quantity for |
| Name of | | | CGB-400 Gel, | Dose Gel, | Vehicle, |
| Ingredient | Function | Grade | % w/w | % w/w | % w/w |
| CG-101 | Active | In-house | 40.00 | 25.00 | — |
| | Ingredient | | | | |
| Propylene | Solvent | USP/NF | 10.00 | 10.00 | 10.00 |
| Glycol | | | | | |
| Hydroxyethyl | Gelling | USP/NF | 1.00 | 1.00 | 1.20 |
| Cellulose | Agent | | | | |
| d-Limonene | Fragrance | Food Grade | 1.00 | 1.00 | 0.20 |
| α-Tocopherol | Antioxidant | USP | 0.05 | 0.05 | 0.05 |
| Methylparaben | Preservative | USP/NF | — | — | 0.20 |
| Propylparaben | Preservative | USP/NF | — | — | 0.05 |
| D&C Yellow | Dye | — | — | — | 7 × 10⁻⁵ |
| No. 10 | | | | | |
| Deionized Water | Solvent | USP | Q.S to 100 | Q.S to 100 | Q.S. to 100 |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A pharmaceutical gel composition, comprising:
an ionic liquid having a cationic component and an anionic component; and
at least one pharmaceutically carrier,
wherein the cationic component comprises choline cation, wherein the anionic component comprises geranate anion, wherein the composition is non-irritating to skin, and wherein the composition comprises about 25% to about 45% by weight of the ionic liquid.

2. The pharmaceutical gel composition of claim 1, wherein the ionic liquid is a deep eutectic solvent, and wherein the deep eutectic solvent has a melting point lower than the melting points of the cationic component and anionic component individually.

3. The pharmaceutical gel composition of claim 1, wherein at least one of the anionic component and cationic component is irritating to the skin when applied in the absence of the other component.

4. The pharmaceutical gel composition of claim 1, wherein the choline cation and the geranate anionic are in a molar ratio ranging from about 1:1 to about 1:2 (cationic component to anionic component).

5. The pharmaceutical gel composition of claim 1, wherein the choline cation and the geranate anionic are in a molar ratio of about 1:2 (cationic component to anionic component).

6. The pharmaceutical gel composition of claim 1, wherein the composition comprises about 40% of the ionic liquid by weight.

7. The pharmaceutical gel composition of claim 1, wherein the composition comprises about 25% of the ionic liquid by weight.

8. The pharmaceutical gel composition of claim 1, wherein the composition further comprises a gelling agent, and wherein the gelling agent is hydroxyethyl cellulose.

9. The pharmaceutical gel composition of claim 1, wherein the composition further comprises from about 0.3% to about 1.5% by weight of hydroxyethyl cellulose.

10. The pharmaceutical gel composition of claim 1, wherein the composition further comprises from about 1.0% by weight of hydroxyethyl cellulose.

11. The pharmaceutical gel composition of claim 1, wherein the composition further comprises from about 5% to about 20% by weight of propylene glycol.

12. The pharmaceutical gel composition of claim 1, wherein the composition further comprises from about 10% by weight of propylene glycol.

13. The pharmaceutical gel composition of claim 1, wherein the composition further comprises an antioxidant, and wherein the antioxidant is α-Tocopherol.

14. The pharmaceutical gel composition of claim 1, wherein the composition further comprises about 0.05% by weight of α-Tocopherol.

15. The pharmaceutical gel composition of claim 1, wherein the composition further comprises from about 35% to about 75% by weight of water.

16. The pharmaceutical gel composition of claim 1, wherein the composition further comprises from about 45% to about 50% by weight of water.

17. The pharmaceutical gel composition of claim 1, wherein the composition further comprises from about 60% to about 65% by weight of water.

18. The pharmaceutical gel composition of any one of claims 1-28, wherein the composition has a viscosity of from about 5,000 cP to about 25,000 cP.

19. The pharmaceutical gel composition of any one of claims 1-29, wherein the composition has a pH of from about 6.0 to about 8.0.

20. The pharmaceutical gel composition of any one of claims 1-30, wherein the composition is stable for up to at least about 6 months at a temperature of about 40° C.

21. The pharmaceutical gel composition of any one of claims 1-31, wherein the composition is stable for up at least about 5 days at 80° C.

22. The pharmaceutical gel composition of any one of claims 1-32, wherein the composition is stable to UV-A light for at least about 3 days.

23. The pharmaceutical gel composition of claim 1, wherein the composition is formulated for topical administration.

24. The pharmaceutical gel composition of claim 23, wherein the composition provides therapeutically effective amount of the ionic liquid to at least one of the dermis or epidermis.

25. The pharmaceutical gel composition of claim 23, for inhibiting a member of the Kallikrein 5 (KLK 5) in a subject in need thereof.

26. The pharmaceutical gel composition of claim 23, for preventing, treating, and/or ameliorating at least one symptom of a KLK-mediated skin disease or disorder in a subject in need thereof.

27. The pharmaceutical gel composition of claim 26, wherein the KLK-mediated skin disease or disorder is a KLK 5-mediated skin disease or disorder.

28. The method of claim 27, wherein the skin disease or disorder is acne or rosacea.

29. The method of claim 27, wherein the skin disease or disorder is rosacea.

30. The method of claim 29, wherein the at least one symptom is redness, lesions, pustules, pruritis, skin irritation, or combinations thereof.
